SEQUENCE LISTING (41 sequences)

SEQ ID NO: 1 - protein, 27 aa, Homo sapiens
MAQVTLKQSGAEVKKPGSSVKVSCTAS

SEQ ID NO: 2 - protein, 10 aa, Homo sapiens
GGTLRTYGVS

SEQ ID NO: 3 - protein, 14 aa, Homo sapiens
WVRQAPGQGLEWLG

SEQ ID NO: 4 - protein, 17 aa, Homo sapiens
RTIPLFGKTDYAQKFQG

SEQ ID NO: 5 - protein, 32 aa, Homo sapiens
RVTITADKSMDTSFMELTSLTSEDTAVYYCAR

SEQ ID NO: 6 - protein, 13 aa, Homo sapiens
DLTTLTSYNWWDL

SEQ ID NO: 7 - protein, 11 aa, Homo sapiens
WGQGTLVTVSS

SEQ ID NO: 8 - protein, 23 aa, Homo sapiens
EIVLTQSPGTLSLSPGERATLSC

SEQ ID NO: 9 - protein, 12 aa, Homo sapiens
RASQSVTSSQLA

SEQ ID NO: 10 - protein, 15 aa, Homo sapiens
WYQQKPGQAPRLLIS

SEQ ID NO: 11 - protein, 7 aa, Homo sapiens
GASNRAT

SEQ ID NO: 12 - protein, 32 aa, Homo sapiens
GIPDRFSGSGSGTDFTLTISRLEPEDFAVYYC

SEQ ID NO: 13 - protein, 8 aa, Homo sapiens
QQYGSSPT

SEQ ID NO: 14 - protein, 13 aa, Homo sapiens
FGGGTKVEIKRAA

SEQ ID NO: 15 - protein, 124 aa, Homo sapiens
MAQVTLKQSGAEVKKPGSSVKVSCTASGGTLRTYGVSWVRQAPGQGLEWLGRTIPLFGKTDYAQKFQGRVTITADKSMDTSFMELTSLTSEDTAVYYCARDLTTLTSYNWWDLWGQGTLVTVSS

SEQ ID NO: 16 - protein, 110 aa, Homo sapiens
EIVLTQSPGTLSLSPGERATLSCRASQSVTSSQLAWYQQKPGQAPRLLISGASNRATGIPDRFSGSGSGTDFTLTISRLEPEDFAVYYCQQYGSSPTFGGGTKVEIKRAA

SEQ ID NO: 17 - protein, 27 aa, Homo sapiens
MAQVQLQQSGAGVKKPGSSVRVSCSAS

SEQ ID NO: 18 - protein, 10 aa, Homo sapiens
GGTLRTYALS

SEQ ID NO: 19 - protein, 14 aa, Homo sapiens
WVRQVPGQGFEWMG

SEQ ID NO: 20 - protein, 17 aa, Homo sapiens
RIIPMFGKTDYAQKFQG

SEQ ID NO: 21 - protein, 32 aa, Homo sapiens
RLSITADKSMDTGFMELTSLTSEDTAVYYCAR

SEQ ID NO: 22 - protein, 13 aa, Homo sapiens
DLTTLTSYNWLDI

SEQ ID NO: 23 - protein, 23 aa, Homo sapiens
ETTLTQSPGTLSLSPGERATLSC

SEQ ID NO: 24 - protein, 12 aa, Homo sapiens
RASQSVSSNYLA

SEQ ID NO: 25 - protein, 15 aa, Homo sapiens
WYQKKPGQAPRLLIY

SEQ ID NO: 26 - protein, 7 aa, Homo sapiens
GASSRAT

SEQ ID NO: 27 - protein, 32 aa, Homo sapiens
GIPDRFSGSGSGTDFTLTINRLEPEDFAVYYC

SEQ ID NO: 28 - protein, 8 aa, Homo sapiens
QQYGRSPT

SEQ ID NO: 29 - protein, 13 aa, Homo sapiens
FGQGTKVEIKRAA

SEQ ID NO: 30 - protein, 124 aa, Homo sapiens
MAQVQLQQSGAGVKKPGSSVRVSCSASGGTLRTYALSWVRQVPGQGFEWMGRIIPMFGKTDYAQKFQGRLSITADKSMDTGFMELTSLTSEDTAVYYCARDLTTLTSYNWLDIWGQGTLVTVSS

SEQ ID NO: 31 - protein, 110 aa, Homo sapiens
ETTLTQSPGTLSLSPGERATLSCRASQSVSSNYLAWYQKKPGQAPRLLIYGASSRATGIPDRFSGSGSGTDFTLTINRLEPEDFAVYYCQQYGRSPTFGQGTKVEIKRAA

SEQ ID NO: 32 - protein, 25 aa, Homo sapiens
QVQLQQSGAEVKKPGSSVKVSCKAS

SEQ ID NO: 33 - protein, 17 aa, Homo sapiens
GTIPLFGKTDYAQKFQG

SEQ ID NO: 34 - protein, 23 aa, Homo sapiens
ETTLTQSPGILSLSPGDRATLSC

SEQ ID NO: 35 - protein, 12 aa, Homo sapiens
RASQSVGSVNLA

SEQ ID NO: 36 - protein, 15 aa, Homo sapiens
WYQQRPGQAPRLLIH

SEQ ID NO: 37 - protein, 32 aa, Homo sapiens
GIPDRFSGVGSGTDFTLTINRLEPDDFAVYYC

SEQ ID NO: 38 - protein, 9 aa, Homo sapiens
QQYGTSPIT

SEQ ID NO: 39 - protein, 11 aa, Homo sapiens
FGQGTRLEIKR

SEQ ID NO: 40 - protein, 122 aa, Homo sapiens
QVQLQQSGAEVKKPGSSVKVSCKASGGTLRTYGVSWVRQAPGQGLEWLGGTIPLFGKTDYAQKFQGRVTITADKSMDTSFMELTSLTSEDTAVYYCARDLTTLTSYNWWDLWGQGTLVTVSS

SEQ ID NO: 41 - protein, 109 aa, Homo sapiens
ETTLTQSPGILSLSPGDRATLSCRASQSVGSVNLAWYQQRPGQAPRLLIHGASNRATGIPDRFSGVGSGTDFTLTINRLEPDDFAVYYCQQYGTSPITFGQGTRLEIKR

(19) United States
(12) Patent Application Publication
(10) Pub. No.: US 2024/0083981 A1
(43) Pub. Date: Mar. 14, 2024

(54) TREATMENT OF HERPES SIMPLEX VIRUS INFECTION

(71) Applicant: United BioPharma, Inc., Hsinchu County (TW)

(72) Inventors: Be-Sheng KUO, Hsinchu County (TW); Chao-Hung LI, Hsinchu County (TW); Hsiao-Yun SHAO, Hsinchu County (TW); Yaw-Jen LIU, Hsinchu County (TW); Shugene LYNN, Hsinchu County (TW); Chang Yi WANG, Hsinchu County (TW)

(73) Assignee: United BioPharma, Inc., Hsinchu County (TW)

(21) Appl. No.: 18/460,246

(22) Filed: Sep. 1, 2023

Related U.S. Application Data
(60) Provisional application No. 63/403,408, filed on Sep. 2, 2022.

(51) Int. Cl. C07K 16/08; A61P 31/22

(52) U.S. Cl. CPC C07K 16/087; A61P 31/22; A61K 2039/55; C07K 2317/24

(57) ABSTRACT

The present invention relates to the treatment of herpes simplex virus (HSV) infection using an anti-HSV antibody. In particular, the anti-HSV antibody specifically binds to the glycoprotein D (gD) of herpes simplex virus-1 (HSV-1) and herpes simplex virus-2 (HSV-2). The treatment of the present invention is effective against drug-resistant and/or recurrent HSV infection.

### RELATED APPLICATION

This application claims priority to U.S. Provisional Application No. 63/403,408, filed on Sep. 2, 2022, the content of which is hereby incorporated by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Aug. 30, 2023, is named “UB0002WO-Seq-List.xml” and is 37,103 bytes in size. The sequence listing contained in this .XML file is part of the specification and is hereby incorporated by reference herein in its entirety.

### TECHNOLOGY FIELD

The present invention relates to the treatment of herpes simplex virus (HSV) infection using an anti-HSV antibody. In particular, the anti-HSV antibody specifically binds to the glycoprotein D (gD) of herpes simplex virus-1 (HSV-1) and herpes simplex virus-2 (HSV-2). The treatment of the present invention is effective against drug-resistant and/or recurrent HSV infection.

### BACKGROUND OF THE INVENTION

Herpes simplex virus types 1 and 2 (HSV-1 and HSV-2), members of the herpesvirus family, are highly prevalent worldwide pathogens that are responsible for recurrent oral and genital ulcerations. Generally, HSV infection does not cause severe symptoms and complications but can cause serious symptoms in some cases of infection in immunocompromised patients and newborns. Although symptoms are less severe in average patients, HSV can still recur in many patients. HSV recurrence can bother those patients for years and reduce their quality of life.

Although there are several licensed antiviral agents for the treatment of primary and recurrent HSV infection, these antivirals are not always effective and can have significant side effects. Moreover, drug-resistant HSV strains (e.g., acyclovir-resistant) significantly reduce the efficacy of these traditional antiviral agents.

Those patients with high recurrent rates or infected by drug-resistant strains lack a good, alternative treatment option. There is a need to provide better therapies for treating recurrent HSV infection, especially those patients with drug-resistant HSV infection. Therefore, new interventions for effective prophylactic and therapeutic purposes are still urgently in demand.

### SUMMARY OF THE INVENTION

The present invention is based on the finding that an anti-HSV antibody is effective against drug-resistant HSV strains and reduces the recurrence of HSV infection. In particular, the anti-HSV antibody specifically binds to the glycoprotein D (gD) of herpes simplex virus-1 (HSV-1) and herpes simplex virus-2 (HSV-2). The anti-HSV antibody can neutralize drug-resistant HSV strains and/or inhibit viral transmission. Treatment with the anti-HSV antibody effectively reduces symptoms caused by drug-resistant HSV infection and reduces or delays the recurrence of HSV infection.

Therefore, in one aspect, the present invention provides a method for treating drug-resistant and/or recurrent herpes simplex virus (HSV) infection comprising administering to a subject in need thereof an anti-HSV antibody, wherein the anti-HSV antibody specifically binds to the glycoprotein D (gD) of herpes simplex virus-1 (HSV-1) and herpes simplex virus-2 (HSV-2).

In some embodiments, the anti-HSV antibody neutralizes drug-resistant HSV strains.

In some embodiments, the anti-HSV antibody inhibits viral transmission.

In some embodiments, the anti-HSV antibody comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13.

In some embodiments, the anti-HSV antibody comprises
the VH comprises the amino acid sequence of SEQ ID NO: 15; and/or
the VL comprises the amino acid sequence of SEQ ID NO: 16.

In some embodiments, the anti-HSV antibody comprises
(a) a heavy chain variable region (VH) which comprises a heavy chain complementary determining region 1 (HC CDR1) comprising the amino acid sequence of SEQ ID NO: 18, a heavy chain complementary determining region 2 (HC CDR2) comprising the amino acid sequence of SEQ ID NO: 20, and a heavy chain complementary determining region 3 (HC CDR3) comprising the amino acid sequence of SEQ ID NO: 22; and
(b) a light chain variable region (VL) which comprises a light chain complementary determining region 1 (LC CDR1) comprising the amino acid sequence of SEQ ID NO: 24, a light chain complementary determining region (LC CDR2) comprising the amino acid sequence of SEQ ID NO: 26, and a light chain complementary determining region 3 (LC CDR3) comprising the amino acid sequence of SEQ ID NO: 28.

In some embodiments, the anti-HSV antibody comprises
the VH comprises the amino acid sequence of SEQ ID NO: 30; and/or
the VL comprises the amino acid sequence of SEQ ID NO: 31.

In some embodiments, the anti-HSV antibody comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 33, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 35, a light chain complementary determining region (LC CDR2) comprising the amino acid sequence of SEQ ID NO: 11, and a light chain complementary determining region 3 (LC CDR3) comprising the amino acid sequence of SEQ ID NO: 38.

In some embodiments, the anti-HSV antibody comprises
the VH comprises the amino acid sequence of SEQ ID NO: 40; and/or
the VL comprises the amino acid sequence of SEQ ID NO: 41.

In some embodiments, the antibody is an antigen-binding fragment thereof

In some embodiments, the antibody is humanized.

In some embodiments, the subject is susceptible to or infected with a drug-resistant HSV strain.

In some embodiments, the subject is a patient having a weakened immune system.

In some embodiments, the anti-HSV antibody is administered to the subject in a single dose.

In some embodiments, the antibody is administered in an amount effective in reducing symptoms caused by the HSV infection

In some embodiments, the antibody is administered in an amount effective in delaying recurrent incidences and/or reducing recurrent frequency.

In some embodiments, the anti-HSV antibody is administered to the subject after symptoms occur.

The present invention also provides an anti-HSV antibody as described herein or a composition comprising the same for use in treating drug-resistant and/or recurrent HSV infection in a subject in need thereof. Further disclosed is the use of an anti-HSV antibody as described herein for manufacturing a medicament for treating drug-resistant and/or recurrent HSV infection in a subject in need thereof.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows the determination of the binding affinity of UB-621 to the recombinant gD by surface plasmon resonance (SPR) analysis.

FIG. 2 shows the potency, IC₅₀, and IC₉₀ values of UB-621 in inhibition of laboratory HSV-1 and HSV-2 strains to Vero cells.

FIGS. 3A and 3B show the therapeutic effects of a single SC dose of UB-621 on genital symptoms (FIG. 3A) and mortality (FIG. 3B) in mice with HSV-2 vaginal infection.

FIGS. 4A and 4B show the therapeutic effects of UB-621 administered to mice after the appearance of infection symptoms. The severity of vaginitis (genital symptoms) is shown in FIG. 4A, and the survival rate (mortality) is shown in FIG. 4B.

FIGS. 5A and 5B show the potency, IC₅₀ values of UB-621 and acyclovir (ACV) in inhibition of the artificial HSV mutant strains resistant to ACV, HSV-1 RE TKnull (FIG. 5A) and HSV-2 333 TKnull (FIG. 5B) in Vero cells. The results show that UB-621 is more potent against infections of both ACV-resistant HSV-1 RE TKnull (A) and HSV-2 333 TKnull (B) mutant strains.

FIGS. 6A-6C show the potency, IC₅₀, and IC₉₀ values of UB-621 and acyclovir (ACV) in inhibition of clinical HSV-1 and HSV-2 isolates, including HSV-1 and HSV-2 from American origins (FIG. 6A), HSV-2 from Asian origins (FIG. 6B), and HSV-2 ACV-resistance (FIG. 6C). The results show that UB-621 is more potent than ACV in inhibiting clinically derived HSV isolates of American and Asian origins, and ACV-resistance.

FIG. 7 shows the susceptibility to inhibition by UB-621 of clinical HSV isolates resistant to ACV (Acyclovir), PFA (Foscarnet), or CDV (Cidofovir).

FIG. 8 shows the therapeutic effects of a single dose of UB-621 against the infection of the ACV-resistant HSV-1 clinical isolate in a murine model.

FIGS. 9A and 9B show the therapeutic effects of UB-621 against the infection of ACV-resistant HSV-2 clinical isolates in genital-infected mice. The effects of UB-621 on survival rates after the HSV-2 clinical isolated IC infection are shown in FIG. 9A. Single-dose of UB-621 increased survival rates against the infection of ACV-resistant HSV-2 IC strain. The effects of UB-621 on the severity of vaginitis induced by the infection of the clinical isolated HSV-2 IC strain are shown in FIG. 9B. Single-dose of UB-621 reduced clinical vaginitis against the infection of ACV-resistant HSV-2 IC strain. * statistical significance with p value <0.05; ** p value <0.01.

FIGS. 10A and 10B show the therapeutic effects of UB-621 against the infection of ACV-resistant HSV-2 clinical isolates in genital-infected mice. The effects of UB-621 on survival rates after the HSV-2 clinical isolated pol-mut infection are shown in FIG. 10A. Single-dose of UB-621 increased survival rates against the infection of ACV-resistant HSV-2 pol-mut strain. The effects of UB-621 on the severity of vaginitis induced by the infection of the clinical isolated HSV-2 pol-mut strain are shown in FIG. 10B. Single-dose of UB-621 reduced clinical vaginitis against the infection of ACV-resistant HSV-2 pol-mut strain. * statistical significance with p value <0.05.

FIGS. 11A and 11B show the inhibition of HSV-1 (FIG. 11A) and HSV-2 (FIG. 11B) cell-to-cell spread by UB-621.

FIG. 12 shows the inhibition effects of UB-621 on HSV-1 anterograde neuron-to-neuron transmission in BALB/c mice. ** statistical significance with p value <0.01

FIGS. 13A and 13B show the significant effects of UB-621 on primary HSV-2 infection in guinea pigs. The effects of UB-621 on the severity of vaginitis in primary infection are shown in FIG. 13A. UB-621 dosing reduced the symptoms of vaginitis in genital HSV-2 infected guinea pigs. The effects of UB-621 on cumulative recurrent numbers are shown in FIG. 13B. UB-621 dosing delayed recurrent incidences and reduced the frequency of recurrence in genital HSV-2-infected guinea pigs.

FIGS. 14A and 14B show the significant effects of UB-621 on recurrent HSV-2 infection in guinea pigs. The effects of UB-621 on the severity of vaginitis after the first HSV recurrence are shown in FIG. 14A. Dosing UB-621 after the first HSV recurrence reduced the symptoms of vaginitis in genital HSV-2 infected guinea pigs. The effects of UB-621 on cumulative recurrent numbers are shown in FIG. 14B. Dosing UB-621 after the first HSV recurrence reduced the frequency of recurrence in genital HSV-2-infected guinea pigs.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, specific terms are first defined. Additional definitions are set forth throughout the detailed description. Unless defined otherwise, all technical and scientific terms used herein have the same meanings as is commonly understood by one of skill in the art to which this invention belongs.

### I. Definitions

In order to provide a clear and ready understanding of the present invention, specific terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including, which means permitting the presence of one or more features, ingredients, or components. The term “comprise” or “comprising” encompasses the term “consists” or “consisting of.”

As used herein, a “herpes virus” refers to members of the herpetoviridae family, including herpes simplex virus type 1 (HSV-1) and herpes simplex virus type 2 (HSV-2). The present invention is particularly advantageous for use to reduce symptoms caused by HSV infections. HSV can infect many areas of the body, including the oral cavity, genital sites, eye, skin, and brain. In general, HSV-1 primarily infects the oral cavity, and HSV-2 primarily infects genital sites. Normally, HSV is transmitted to a non-infected subject by direct contact with the infected site of the infected subject. Clinical symptoms associated with HSV infection are well known in this art. HSV infections may be asymptomatic or symptomatic which may include painful blisters or ulcers and can recur over time. HSV recurrence can be triggered by several factors such as stress, immunodeficiency, sunburn, and injury. HSV infections can be fatal, especially in individuals with immunodeficiency. Typical HSV infectious diseases include herpetic gingivostomatitis, herpes labialis, herpes eye infection (herpes keratitis), herpes genitalis, herpetic whitlow, herpes gladiatorum, herpesviral encephalitis, herpesviral meningitis, and herpes esophagitis. Initial symptoms of orolabial herpes include painful vesicular eruptions with distinct vesicles appearing on the lips, tongue, or buccal mucosa. The vesicles can coalesce and rupture to form shallow ulcers covered with necrotic material. Typical clinical symptoms for genital herpes include small, grouped vesicles on erythematous bases. The vesicles can unroof spontaneously or by direct abrasion to form ulcerations. The ulcerations typically form a crust and then undergo re-epithelialization.

See U.S. Publication No. US20020147210A1 and U.S. Pat. No. 6,599,945B2, for example, the entire content of which is incorporated herein by reference.

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, 20, or 10 amino acid residues).

As used herein, the term “approximately” or “about” refers to a degree of acceptable deviation that will be understood by persons of ordinary skill in the art, which may vary to some extent depending on the context in which it is used. Specifically, “approximately” or “about” may mean a numeric value having a range of ±10% or ±5% or ±3% around the cited value.

As used herein, the term “substantially identical” refers to two sequences having 80% or more, preferably 85% or more, more preferably 90% or more, and even more preferably 95% or more homology.

As used herein, the term “antibody” (interchangeably used in plural form, antibodies) means an immunoglobulin molecule having the ability to specifically bind to a particular target antigenic molecule. As used herein, the term “antibody” includes not only intact (i.e., full-length) antibody molecules but also antigen-binding fragments thereof retaining antigen binding ability (e.g., Fab, Fab′, F(ab′)2, and Fv). Such fragments are also well known in the art and are regularly employed both in vitro and in vivo. The term “antibody” also includes chimeric antibodies, humanized antibodies, human antibodies, diabodies, linear antibodies, single chain antibodies, multispecific antibodies (e.g., bispecific antibodies), and any other modified configuration of the immunoglobulin molecule that comprises an antigen recognition site of the required specificity, including amino acid sequence variants of antibodies, glycosylation variants of antibodies, and covalently modified antibodies.

An intact or complete antibody comprises two heavy chains and two light chains. Each heavy chain contains a variable region (V_{H}) and a first, second, and third constant regions (C_{H}1, C_{H}2, and C_{H}3); and each light chain contains a variable region (V_{L}) and a constant region (C_{L}). The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light chains and those of heavy chains are responsible for antigen binding. The variables regions in both chains are responsible for antigen binding generally, each of which contains three highly variable regions, called the complementarity determining regions (CDRs); namely, heavy (H) chain CDRs including HC CDR1, HC CDR2, HC CDR3, and light (L) chain CDRs including LC CDR1, LC CDR2, and LC CDR3. The three CDRs are franked by framework regions (FR1, FR2, FR3, and FR4), which are more highly conserved than the CDRs and form a scaffold to support the hypervariable regions. The constant regions of the heavy and light chains are not responsible for antigen binding but are involved in various effector functions. Depending on the antibody amino acid sequence of the constant domain of its heavy chains, immunoglobulins can be assigned to different classes. There are five major classes of immunoglobulins: IgA, IgD, IgE, IgG, and IgM. The heavy-chain constant domains that correspond to the different classes of immunoglobulins are called alpha, delta, epsilon, gamma, and mu, respectively.

As used herein, the term “antigen-binding fragment” or “antigen-binding domain” refers to a portion or region of an intact antibody molecule that is responsible for antigen binding. An antigen-binding fragment is capable of binding to the same antigen to which the parent antibody binds. Examples of antigen-binding fragments include, but are not limited to: (i) a Fab fragment, which can be a monovalent fragment composed of a V_{H}-C_{H}1 chain and a V_{L}-C_{L} chain; (ii) an F(ab′)2 fragment which can be a bivalent fragment composed of two Fab fragments linked by a disulfide bridge at the hinge region; (iii) an Fv fragment, composed of the V_{H} and V_{L} domains of an antibody molecule associated together by noncovalent interaction; (iv) a single chain Fv (scFv), which can be a single polypeptide chain composed of a V_{H} domain and a V_{L} domain via a peptide linker; and (v) an (scFv)₂, which can contain two V_{H} domains linked by a peptide linker and two V_{L} domains, which are associated with the two V_{H} domains via disulfide bridges.

As used herein, the term “chimeric antibody” refers to an antibody containing polypeptides from different sources, e.g., different species. In some embodiments, in chimeric antibodies, the variable region of both light and heavy chains may mimic the variable region of antibodies derived from one species of mammals (e.g., a non-human mammal such as mouse, rabbit, and rat), while the constant region may be homologous to the sequences in antibodies derived from another mammal such as a human.

As used herein, the term “humanized antibody” refers to an antibody comprising a framework region originated from a human antibody and one or more CDRs from a non-human (usually a mouse or rat) immunoglobulin.

As used herein, the term “human antibody” refers to an antibody in which essentially the entire sequences of the light chain and heavy chain sequences, including the complementary determining regions (CDRs), are from human genes. In some circumstances, the human antibodies may include one or more amino acid residues not encoded by human germline immunoglobulin sequences (e.g., by mutations in one or more of the CDRs) or in one or more of the FRs, such as to, for example, decrease possible immunogenicity, increase affinity, and eliminate cysteines that might cause undesirable folding.

As used herein, the term “specific binds” or “specifically binding” refers to a non-random binding reaction between two molecules, such as the binding of the antibody to an epitope of its target antigen. An antibody that “specifically binds” to a target antigen or an epitope is a term well understood in the art, and methods to determine such specific binding are also well known in the art. An antibody “specifically binds” to a target antigen if it binds with greater affinity/avidity, more readily, and/or greater duration than it binds to other substances. In other words, it is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, the affinity of the binding can be defined in terms of a dissociation constant (K_{D}). Typically, specific binding, when used with respect to an antibody, can refer to an antibody that specifically binds to (recognize) its target with a KD value less than about 10⁻⁸ M, such as about 10⁻⁹ M or less, about 10⁻¹⁰ M or less, about 10⁻¹¹ M or less, about 10⁻¹² M or less, or even less, and binds to the specific target with an affinity corresponding to a K_{D} that is at least ten-fold lower than its affinity for binding to a non-specific antigen (such as BSA or casein), such as at least 100 fold lower, e.g., at least 1,000 fold lower or at least 10,000 fold lower.

As used herein, the term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to DNA that is complementary or identical to an mRNA, in either single-stranded or double-stranded form.

As used herein, the term “complementary” refers to the topological compatibility or matching together of interacting surfaces of two polynucleotides. A first polynucleotide is complementary to a second polynucleotide when the nucleotide sequence of the first polynucleotide is identical to the nucleotide sequence of the polynucleotide binding partner of the second polynucleotide. Thus, the polynucleotide whose sequence 5′-ATATC-3′ is complementary to a polynucleotide whose sequence is 5′-GATAT-3′.”

As used herein, the term “encoding” refers to the natural property of specific sequences of nucleotides in a polynucleotide (e.g., a gene, a cDNA, or an mRNA) to serve as templates for synthesis of other polymers and macromolecules in biological processes having either a given sequence of RNA transcripts (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

As used herein, the term “recombinant nucleic acid” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence (expression vector) or maintaining the given nucleotide sequence for replicating it, manipulating it, or transferring it between different locations (e.g., between different organisms). Vectors can be introduced into a suitable host cell for the above-described purposes. A “recombinant cell” refers to a host cell that has introduced a recombinant nucleic acid into it. “A transformed cell” means a cell into which has been introduced, by means of recombinant DNA techniques, a DNA molecule encoding a protein of interest.

Vectors may be of various types, including plasmids, cosmids, episomes, fosmids, artificial chromosomes, phages, viral vectors, etc. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example, and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, a secretion signal sequence (e.g., α-mating factor signal), a stop codon, and other control sequence (e.g., Shine-Dalgarno sequences and termination sequences). Preferably, vectors may further contain a marker sequence (e.g., an antibiotic-resistant marker sequence) for the subsequent screening/selection procedure. For the purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes a tag for the purpose of purification (e.g., a His-tag).

As used herein, the term “treatment” refers to the application or administration of one or more active agents to a subject afflicted with a disorder, a symptom or condition of the disorder, or a progression of the disorder, with the purpose to cure, heal, alleviate, relieve, alter, remedy, ameliorate, improve, or affect the disorder, the symptom or condition of the disorder, the disabilities induced by the disorder, or the progression or predisposition of the disorder.

### II. Antibodies Directed to HSV

According to the present invention, an anti-HSV antibody as used herein specifically binds to gD of HSV-1 and HSV-2 and is capable of neutralizing drug-resistant HSV strains. Neutralization of virus typically occurs when an antibody binds to the virus and thus prevents infection of a susceptible cell. It is found that the anti-HSV antibody of the present invention exhibits high potency in neutralizing drug-resistant HSV strains. The potency of neutralization may be measured in a method known in the art. For example, the anti-HSV antibody of the present invention may have an IC50 less than 100 nM, for example, from about 1 nM to about 99 nM, such as from about 1 nM to about 80 nM, about 1 nM to about 70 nM, about 1 nM to about 60 nM, about 1 nM to about 50 nM, about 1 nM to about 40 nM, about 1 nM to about 30 nM, about 1 nM to about 20 nM, or about 1 nM to about 10 nM, in a viral plaque assay.

It is also found that an anti-HSV antibody as used herein inhibits viral transmission, including cell-to-cell and/or neuron-to-neuron transmission.

Exemplary anti-HSV antibodies include monoclonal antibody (mAb) E317 (i.e., UB-621 mAb described in the examples below), E425, and Y571 as described in U.S. Pat. No. 8,252,906, which is herein incorporated by reference in its entirely.

According to U.S. Pat. No. 8,252,906, mAb E317, E425, and Y571 each comprises a heavy chain variable region (V_{H}) having complementary determining regions thereof (HC CDR1, HC CDR2, and HC CDR3) and a light chain variable region (V_{L}) having complementary determining regions thereof (LC CDR1, LC CDR2, and LC CDR3) as shown in Table 1.

TABLE 1
Amino acid sequences of mAb E317, E425, and Y571.
E317
V_{H} domain

| FW1 | CDR1 | FW2 | CDR2 |
|---|---|---|---|
| MAQVTLKQSGAEVKKPG | GGTLRTYGVS | WVRQAPGQGLEWLG | RTIPLFGKTDYA |
| SSVKVSCTAS | (SEQ ID NO: 2) | (SEQ ID NO: 3) | QKFQG |
| (SEQ ID NO: 1) | | | (SEQ ID NO: 4) |

| FW3 | CDR3 | FW4 |
|---|---|---|
| RVTITADKSMDTSFMELTS | DLTTLTSYNW | WGQGTLVTVSS |
| LTSEDTAVYYCAR | WDL | (SEQ ID NO: 7) |
| (SEQ ID NO: 5) | (SEQ ID NO: 6) | |

V_{L} domain

| FW1 | CDR1 | FW2 | CDR2 |
|---|---|---|---|
| EIVLTQSPGTLSLSPGERA | RASQSVTSSQL | WYQQKPGQAPRLLIS | GASNRAT |
| TLSC | A | (SEQ ID NO: 10) | (SEQ ID NO: 11) |
| (SEQ ID NO: 8) | (SEQ ID NO: 9) | | |

| FW3 | CDR3 | FW4 |
|---|---|---|
| GIPDRFSGSGSGTDFTLTIS | QQYGSSPT | FGGGTKVEIKRAA |
| RLEPEDFAVYYC | (SEQ ID NO: 13) | (SEQ ID NO: 14) |
| (SEQ ID NO: 12) | | |

Full-length amino acid sequences of heavy chain and light chain

| | |
|---|---|
| heavy chain | MAQVTLKQSGAEVKKPGSSVKVSCTASGGTLRTYGVSWVRQAPGQGLEWLGRTI |
| | PLFGKTDYAQKFQGRVTITADKSMDTSFMELTSLTSEDTAVYYCARDLTTLTSYN |
| | WWDLWGQGTLVTVSS |
| | (SEQ ID NO: 15) |
| light chain | EIVLTQSPGTLSLSPGERATLSCRASQSVTSSQLAWYQQKPGQAPRLLISGASNRAT |
| | GIPDRFSGSGSGTDFTLTISRLEPEDFAVYYCQQYGSSPTFGGGTKVEIKRAA |
| | (SEQ ID NO: 16) |

E425
V_{H} domain

| FW1 | CDR1 | FW2 | CDR2 |
|---|---|---|---|
| MAQVQLQQSGAGVKKPG | GGTLRTYALS | WVRQVPGQGFEWMG | RIIPMFGKTDYA |
| SSVRVSCSAS | (SEQ ID NO: 18) | (SEQ ID NO: 19) | QKFQG |
| (SEQ ID NO: 17) | | | (SEQ ID NO: 20) |

| FW3 | CDR3 | FW4 |
|---|---|---|
| RLSITADKSMDTGFMELT | DLTTLTSYNWL | WGQGTLVTVSS |
| SLTSEDTAVYYCAR | DI | (SEQ ID NO: 7) |
| (SEQ ID NO: 21) | (SEQ ID NO: 22) | |

V_{L} domain

| FW1 | CDR1 | FW2 | CDR2 |
|---|---|---|---|
| ETTLTQSPGTLSLSPGERA | RASQSVSSNYL | WYQKKPGQAPRLLIY | GASSRAT |
| TLSC | A | (SEQ ID NO: 25) | (SEQ ID NO: 26) |
| (SEQ ID NO: 23) | (SEQ ID NO: 24) | | |

| FW3 | CDR3 | FW4 |
|---|---|---|
| GIPDRFSGSGSGTDFTLTI | QQYGRSPT | FGQGTKVEIKRAA |
| NRLEPEDFAVYYC | (SEQ ID NO: 28) | (SEQ ID NO: 29) |
| (SEQ ID NO: 27) | | |

Full-length amino acid sequences of heavy chain and light chain

| | |
|---|---|
| heavy chain | MAQVQLQQSGAGVKKPGSSVRVSCSASGGTLRTYALSWVRQVPGQGFEWMGRII |
| | PMFGKTDYAQKFQGRLSITADKSMDTGFMELTSLTSEDTAVYYCARDLTTLTSYN |
| | WLDIWGQGTLVTVSS |
| | (SEQ ID NO: 30) |
| light chain | ETTLTQSPGTLSLSPGERATLSCRASQSVSSNYLAWYQKKPGQAPRLLIYGASSRAT |
| | GIPDRFSGSGSGTDFTLTINRLEPEDFAVYYCQQYGRSPTFGQGTKVEIKRAA |
| | (SEQ ID NO: 31) |

Y571
V_{H} domain

| FW1 | CDR1 | FW2 | CDR2 |
|---|---|---|---|
| QVQLQQSGAEVKKPGSS | GGTLRTYGVS | WVRQAPGQGLEWLG | GTIPLFGKTDYA |
| VKVSCKAS | (SEQ ID NO: 2) | (SEQ ID NO: 3) | QKFQG |
| (SEQ ID NO: 32) | | | (SEQ ID NO: 33) |

| FW3 | CDR3 | FW4 |
|---|---|---|
| RVTITADKSMDTSFMELTS | DLTTLTSYNW | WGQGTLVTVSS |
| LTSEDTAVYYCAR | WDL | (SEQ ID NO: 7) |
| (SEQ ID NO: 5) | (SEQ ID NO: 6) | |

V_{L} domain

| FW1 | CDR1 | FW2 | CDR2 |
|---|---|---|---|
| ETTLTQSPGILSLSPGDRA | RASQSVGSVNL | WYQQRPGQAPRLLIH | GASNRAT |
| TLSC | A | (SEQ ID NO: 36) | (SEQ ID NO: 11) |
| (SEQ ID NO: 34) | (SEQ ID NO: 35) | | |

| FW3 | CDR3 | FW4 |
|---|---|---|
| GIPDRFSGVGSGTDFTLTI | QQYGTSPIT | FGQGTRLEIKR |
| NRLEPDDFAVYYC | (SEQ ID NO: 38) | (SEQ ID NO: 39) |
| (SEQ ID NO: 37) | | |

Full-length amino acid sequences of heavy chain and light chain

| | |
|---|---|
| heavy chain | QVQLQQSGAEVKKPGSSVKVSCKASGGTLRTYGVSWVRQAPGQGLEWLGGTIP |
| | LFGKTDYAQKFQGRVTITADKSMDTSFMELTSLTSEDTAVYYCARDLTTLTSYNW |
| | WDLWGQGTLVTVSS |
| | (SEQ ID NO: 40) |
| light chain | ETTLTQSPGILSLSPGDRATLSCRASQSVGSVNLAWYQQRPGQAPRLLIHGASNRA |
| | TGIPDRFSGVGSGTDFTLTINRLEPDDFAVYYCQQYGTSPITFGQGTRLEIKR |
| | (SEQ ID NO: 41) |

In some embodiments, the anti-HSV antibody of the present invention is a functional variant of mAb E317 which is characterized in comprising (a) a VH comprising HC CDR1 of SEQ ID NO: 2, HC CDR2 of SEQ ID NO: 4, and HC CDR3 of SEQ ID NO: 6; and (b) a V_{L} comprising LC CDR1 of SEQ ID NO: 9, LC CDR2 of SEQ ID NO: 11, and HC CDR3 of SEQ ID NO: 13, or an antigen-binding fragment thereof.

In some embodiments, the anti-HSV antibody of the present invention, having (a) a V_{H} comprising HC CDR1 of SEQ ID NO: 2, HC CDR2 of SEQ ID NO: 4, and HC CDR3 of SEQ ID NO: 6; and (b) a V_{L} comprising LC CDR1 of SEQ ID NO: 9, LC CDR2 of SEQ ID NO: 11, and HC CDR3 of SEQ ID NO: 13, can comprise a V_{H} comprising SEQ ID NO: 15 or an amino acid sequence substantially identical thereto and a V_{L} comprising SEQ ID NO: 16 or an amino acid sequence substantially identical thereto. Specifically, the anti-HSV antibody of the present invention includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO:15, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO:16.

In some embodiments, the anti-HSV antibody of the present invention is a functional variant of mAb E425 which is characterized in comprising (a) a VH comprising HC CDR1 of SEQ ID NO: 18, HC CDR2 of SEQ ID NO: 20, and HC CDR3 of SEQ ID NO: 22; and (b) a V_{L} comprising LC CDR1 of SEQ ID NO: 24, LC CDR2 of SEQ ID NO: 26, and HC CDR3 of SEQ ID NO: 28, or an antigen-binding fragment thereof.

In some embodiments, the anti-HSV antibody of the present invention, having (a) a VH comprising HC CDR1 of SEQ ID NO: 18, HC CDR2 of SEQ ID NO: 20, and HC CDR3 of SEQ ID NO: 22; and (b) a V_{L} comprising LC CDR1 of SEQ ID NO: 24, LC CDR2 of SEQ ID NO: 26, and HC CDR3 of SEQ ID NO: 28, can comprise a V_{H} comprising SEQ ID NO: 30 or an amino acid sequence substantially identical thereto and a V_{L} comprising SEQ ID NO: 31 or an amino acid sequence substantially identical thereto. Specifically, the anti-HSV antibody of the present invention includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO: 30, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO: 31.

In some embodiments, the anti-HSV antibody of the present invention is a functional variant of mAb Y571 which is characterized in comprising (a) a VH comprising HC CDR1 of SEQ ID NO: 2, HC CDR2 of SEQ ID NO: 33, and HC CDR3 of SEQ ID NO: 6; and (b) a V_{L} comprising LC CDR1 of SEQ ID NO: 35, LC CDR2 of SEQ ID NO: 11, and HC CDR3 of SEQ ID NO: 38, or an antigen-binding fragment thereof.

In some embodiments, the anti-HSV antibody of the present invention, having (a) a VH comprising HC CDR1 of SEQ ID NO: 2, HC CDR2 of SEQ ID NO: 33, and HC CDR3 of SEQ ID NO: 6; and (b) a V_{L} comprising LC CDR1 of SEQ ID NO: 35, LC CDR2 of SEQ ID NO: 11, and HC CDR3 of SEQ ID NO: 38, can comprise a V_{H} comprising SEQ ID NO: 40 or an amino acid sequence substantially identical thereto and a V_{L} comprising SEQ ID NO: 41 or an amino acid sequence substantially identical thereto. Specifically, the anti-HSV antibody of the present invention includes a V_{H} comprising an amino acid sequence has at least 80% (e.g. 82%, 84%, 85%, 86%, 88%, 90%, 92%, 94%, 95%, 96%, 98%, or 99%) identity to SEQ ID NO: 40, and a V_{L} comprising an amino acid sequence has at least 80% (e.g. 82%, 84%, 85%, 86%, 88%, 90%, 92%, 94%, 95%, 96%, 98%, or 99%) identity to SEQ ID NO: 41.

The anti-HSV antibody of the present invention also includes any recombinantly (engineered)-derived antibody encoded by the polynucleotide sequence encoding the relevant V_{H} or V_{L} amino acid sequences as described herein.

The term “substantially identical” can mean that the relevant amino acid sequences (e.g., in FRs, CDRs, V_{H}, or V_{L}) of a variant differ insubstantially as compared with a reference antibody such that the variant has substantially similar binding activities (e.g., affinity, specificity, or both) and bioactivities relative to the reference antibody. Such a variant may include minor amino acid changes. It is understandable that a polypeptide may have a limited number of changes or modifications that may be made within a certain portion of the polypeptide irrelevant to its activity or function and still result in a variant with an acceptable level of equivalent or similar biological activity or function. In some examples, the amino acid residue changes are conservative amino acid substitution, which refers to the amino acid residue of a similar chemical structure to another amino acid residue, and the polypeptide function, activity, or other biological effect on the properties smaller or substantially no effect. Typically, relatively more substitutions can be made in FR regions, in contrast to CDR regions, as long as they do not adversely impact the binding function and bioactivities of the antibody (such as reducing the binding affinity by more than 50% as compared to the original antibody). In some embodiments, the sequence identity can be about 80%, 82%, 84%, 85%, 86%, 88%, 90%, 92%, 94%, 95%, 96%, 98%, or 99%, or higher, between the reference antibody and the variant. Variants can be prepared according to methods for altering polypeptide sequence known to one of ordinary skills in the art such as those found in references which compile such methods, e.g., Molecular Cloning: A Laboratory Manual, J. Sambrook, et al., eds., Second Edition, Cold Spring Harbor Laboratory Press, Cold Spring Harbor, New York, 1989. For example, conservative substitutions of amino acids include substitutions made amongst amino acids within the following groups: (i) A, G; (ii) S, T; (iii) Q, N; (iv) E, D; (v) M, I, L, V; (vi) F, Y, W; and (vii) K, R, H.

The antibodies described herein may be animal antibodies (e.g., mouse-derived antibodies), chimeric antibodies (e.g., mouse-human chimeric antibodies), humanized antibodies, or human antibodies. The antibodies described herein may also include their antigen-binding fragments, e.g., a Fab fragment, a F(ab′)2 fragment, an Fv fragment, a single chain Fv (scFv), and an (scFv)₂. The antibodies or their antigen-binding fragments can be prepared by methods known in the art.

### III. Preparation of Antibodies

Numerous methods conventional in this art are available for obtaining antibodies or antigen-binding fragments thereof.

In some embodiments, the antibodies provided herein may be made by the conventional hybridoma technology. In general, a target antigen optionally coupled to a carrier protein and/or mixed with an adjuvant may be used to immunize a host animal for generating antibodies binding to that antigen. Lymphocytes secreting monoclonal antibodies are harvested and fused with myeloma cells to produce hybridoma. Hybridoma clones formed in this manner are then screened to identify and select those that secrete the desired monoclonal antibodies.

In some embodiments, the antibodies provided herein may be prepared via recombinant technology. In related aspects, isolated nucleic acids that encode the disclosed amino acid sequences, together with vectors comprising such nucleic acids and host cells transformed or transfected with the nucleic acids, are also provided.

For examples, nucleic acids comprising nucleotide sequences encoding the heavy and light chain variable regions of such an antibody can be cloned into expression vectors (e.g., a bacterial vector such as an E. coli vector, a yeast vector, a viral vector, or a mammalian vector) via routine technology, and any of the vectors can be introduced into suitable cells (e.g., bacterial cells, yeast cells, plant cells, or mammalian cells) for expression of the antibodies. Examples of mammalian host cell lines are human embryonic kidney line (293 cells), baby hamster kidney cells (BHK cells), Chinese hamster ovary cells (CHO cells), African green monkey kidney cells (Vero cells), and human liver cells (Hep G2 cells). The recombinant vectors for the expression of the antibodies described herein typically contain a nucleic acid encoding the antibody amino acid sequences operably linked to a promoter, either constitutive or inducible. Typical vectors contain transcription and translation terminators, initiation sequences, and promoters useful for regulation of the expression of the nucleic acid encoding the antibody. The vectors optionally contain selection markers for both prokaryotic and eukaryotic systems. In some examples, both the heavy and light chain coding sequences are included in the same expression vector. In other examples, each of the heavy and light chains of the antibody is cloned into an individual vector and produced separately, which can be then incubated under suitable conditions for antibody assembly.

The recombinant vectors for the expression of the antibodies described herein typically contain a nucleic acid encoding the antibody amino acid sequences operably linked to a promoter, either constitutive or inducible. The recombinant antibodies can be produced in prokaryotic or eukaryotic expression systems, such as bacteria, yeast, insect, and mammalian cells. Typical vectors contain transcription and translation terminators, initiation sequences, and promoters useful for regulation of the expression of the nucleic acid encoding the antibody. The vectors optionally contain selection markers for both prokaryotic and eukaryotic systems. The antibody protein as produced can be further isolated or purified to obtain substantially homogeneous preparations for further assays and applications. Suitable purification procedures, for example, may include fractionation on immunoaffinity or ion-exchange columns, ethanol precipitation, sodium dodecyl sulfate polyacrylamide gel electrophoresis (SDS-PAGE), high-performance liquid chromatography (HPLC), ammonium sulfate precipitation, and gel filtration.

When a full-length antibody is desired, coding sequences of any of the V_{H} and V_{L} chains described herein can be linked to the coding sequences of the Fc region of an immunoglobulin, and the resultant gene encoding a full-length antibody heavy and light chains can be expressed and assembled in a suitable host cell, e.g., a plant cell, a mammalian cell, a yeast cell, or an insect cell.

Antigen-binding fragments can be prepared via routine methods. For example, F(ab′)₂ fragments can be generated by pepsin digestion of a full-length antibody molecule, and Fab fragments can be made by reducing the disulfide bridges of F(ab′)₂ fragments. Alternatively, such fragments can also be prepared via recombinant technology by expressing the heavy and light chain fragments in suitable host cells and having them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

### IV. Compositions

According to the present invention, the anti-HSV antibody may be formulated with a pharmaceutically acceptable carrier into a composition for the purpose of delivery and absorption.

As used herein, “pharmaceutically acceptable” means that the carrier is compatible with an active ingredient in the composition, and preferably can stabilize said active ingredient and is safe to the receiving individual. Said carrier may be a diluent, vehicle, excipient, or matrix to the active ingredient. Typically, a composition comprising an anti-HSV antibody, as described herein as an active ingredient, can be in a form of a solution such as an aqueous solution (e.g., a saline solution, or it can be provided in powder form). The composition may further contain pharmaceutically acceptable auxiliary substances as required to approximate physiological conditions, for example, pH adjusting and buffering agents, such as sodium acetate, sodium chloride, potassium chloride, calcium chloride, sodium lactate and the like. The form of the composition may be suspensions, lotions, solutions, sterilized injection fluid, and packaged powder. The composition of the present invention may be delivered via any physiologically acceptable route, such as parenteral (e.g., intramuscular, intravenous, subcutaneous, and intraperitoneal) and intranasal methods. In certain embodiments, the composition of the present invention is administered as a liquid injectable formulation which can be provided as a ready-to-use dosage form or as a reconstitutable stable powder.

### V. Treatment

The present invention provides a method for treating drug-resistant and/or recurrent HSV infection by administering an anti-HSV antibody as described herein. The method of the present invention is effective in reducing symptoms caused by the HSV infection, delaying recurrent incidences, and/or reducing recurrent frequency.

As used herein, the term “drug-resistance” can indicate that virus is able to survive when exposing to one or more anti-viral drugs. Specifically, drug resistance to an anti-viral drug can indicate that virus replication is not inhibited by a normal amount of the anti-viral drug or clinical efficacy of the agent against the virus has not been shown. The term “multiple-drug resistant” may refer to a virus that is resistant to more than one anti-viral drug.

In some embodiments, the drug-resistant HSV strain has a mutation in tyrosine kinase (TK) or DNA polymerase enzyme. In some embodiments, the strain is resistant to at least one selected from the group consisting of acyclovir (ACV), famciclovir (FCV), penciclovir (PCV), valacyclovir (VCV), trifluridine (TFD), foscarnet (phosphono-formic acid (PFA)) and cidofovir (CDV). In some embodiment, the strain includes clinical isolates such as HSV-1 RH (US origin), HSV-1 Bethesda (US origin), HSV-2 MO (US origin), HSV-2 Bethesda (US origin), HSV-2 JA-1 (Asian origin), HSV-2 JA-2 (Asian origin), HSV-2 JA-3 (Asian origin), HSV-2 poly-mut (polymerase mutant), HSV-2 C7 (TK mutant), C8 (TK mutant), IC (ACV-resistant). In some embodiments, the strain includes clinic isolates (German origin) such as HSV-1 R2, R4, R7, R8, R9, R10, R11, and R13, and HSV-2 R5, R6, and R14.

The method of the present invention using an anti-HSV antibody as described herein is effective against drug-resistant HSV infections. In some embodiments, an anti-HSV antibody as described herein achieves a low IC₅₀ against several drug-resistant HSV strains (such as from 1 nM to 30 nM), having a potency 3-fold or more (e.g., 5-fold, 10-fold, 20-fold, 50-fold, 100-fold, 250-fold, 500-fold, 1,000-fold, 2,500-fold, 5,000-fold or more) greater than acyclovir which provides a high IC₅₀ against several drug-resistant HSV strains (such as from 100 nM to 35,000 or more). In some embodiments, administration of an anti-HSV antibody as described herein is effective in protecting animals with drug-resistant HSV infection from death in an amount of 15 to 50 mg/kg with a single dose.

As used herein, the term “reducing symptoms” or “symptomatic reduction” may mean an active agent which reduces or eliminates one or more symptoms of a disease or other abnormal state. The symptomatic severity level of a disease may be determined by any suitable index or score known in the art. In general, a higher level of the index or a higher score indicates greater disease severity. In some embodiments, animals with HSV infection observed for clinical disease and the clinical symptoms can be scored 0 for no lesion; 1 for erythema only; 2 for single or few vesicles; 3 for ulcerated lesions, and 4 for abnormal movement in addition to lesions at score 3. In some embodiments, animals with HSV infection observed for clinical disease and the clinical symptoms can be scored 0 for normal; 1 for slight redness of external genitalia; 2 for swelling and redness of external genitalia, and/or pus/mucus; 3 for severe swelling of external genitalia with pus/mucus and some alopecia in the surrounding area; 4 for ulceration of genital tissue, redness, and swelling; 5 for increased ulceration, redness, swelling, and hind limb paralysis; and 6 for death. In some embodiments, symptomatic reduction may include a reduced level of such index or a decreased score, for example, by 5%, 10%, 15%, 20%, 25%, 30%, 45%, 50%, 55%, 60%, 65%, 70%, 75%, 80%, 85%, 90%, 95% or more, compared with a diseased level of the index or a diseased score that one of ordinary skill in the art and/or a medical professional, e.g., a doctor would expect a diseased individual or population of similar physical characteristics and medical history to have. In some embodiments, symptomatic reduction may mean that one or more perceived symptoms of a disease or other abnormal state are alleviated to a normal status.

As used herein, “recurrent infection” can mean that infection occur again or repeatedly, for example, second, third or subsequent episode of infection, in a patient, after the previous/initial episode of infection is deemed cured. Normally, the first time that symptoms occur is called a primary or initial outbreak while subsequent outbreaks are called recurrence. In clinical, recurrent HSV infection can indicate reactivation of HSV that is the same in the lesion as the antibodies in the serum. Patient undergoing recurrent infection may mean the virus reactive again. In particular embodiments, some patients with genital herpes may experience one or more recurrences per month; some have a recurrence every two to four months, and some have recurrences less than once every four months. In particular embodiments, some patients with oral herpes have a recurrence one or more times per month, some have a recurrence two to four months, and some have a recurrence less than once every four months.

As used herein, the term “treating recurrent HSV infection” may refer to a reduction in severity, frequency, duration, and/or quantity of one or more recurrent viral symptoms in an infected individual, or a reduction in the mean or median severity, frequency, duration and/or quantity of one or more recurrent viral symptoms for a population of individuals. In some embodiments, administration of an anti-HSV antibody as described herein is effective in delaying incidences of recurrence. In some embodiments, administration of an anti-HSV antibody as described herein is effective in decreasing recurrent numbers or frequency. In certain examples, when an anti-HSV antibody as described herein is administered to a population of patients with herpetic lesions, after cessation of treatment, clinical symptoms do not incur for a median time of at least one month. Preferably, the recurrence-free time is more than 2-3 months.

The term “effective amount” used herein refers to the amount of an active ingredient to confer a desired biological effect in a treated subject or cell. For example, an effective amount as described herein may be an amount of an anti-HSV antibody as an active agent that can provide symptom reduction and/or delayed incidences of recurrence for HSV infection. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience.

In some embodiments, an anti-HSV antibody as described herein is administered in a dose range from 0.01 to 100 mg, particularly 0.1 to 100 mg, more particularly 1 to 80 mg, and even more particularly 10 to 50 mg per kilogram of body weight of the subject. In certain examples, an anti-HSV antibody as described herein is administered in a dose range from 1 to 10 mg/kg e.g. 1 mg/kg, 2 mg/kg, 3 mg/kg, 4 mg/kg, 5 mg/kg, 6 mg/kg, 7 mg/kg, 8 mg/kg, 9 mg/kg or 10 mg/kg.

In some embodiments, the antibody is included in a composition in an entire dose and is administered to the subject in a single dose. In general, the earlier administration of the antibody the better. In some embodiments, the antibody is administered early in the course of the infection, for example, within 10 days (e.g., 10 days, 9 days, 8 days, 7 days, 6 days, 5 days, 4 days, 3 days, 2 days or 1 day) post-infection. Preferably, the antibody is administered within 5 days (e.g., 5 days, 4 days, 3 days, 2 days, or 1 day) post-infection

A subject to be treated by the method of treatment as described herein can be a mammal, more preferably a human. Mammals include, but are not limited to, farm animals, sport animals, pets, primates, horses, dogs, cats, mice, and rats. A human subject who needs the treatment may be a human patient having, at risk for, or suspected of having a target disease/disorder. A subject suspected of having any of such target disease/disorder might show one or more symptoms of the disease/disorder. A subject at risk for the disease/disorder can be a subject having one or more of the risk factors for that disease/disorder. In some embodiments, a subject is a patient infected with HSV or a patient having a weakened immune system, such as infants, pregnant women, cancer patients, organ transplant recipients, and human immunodeficiency virus (HIV) carriers. In some embodiments, a subject is a patient infected with HSV with poor control by small molecular drugs.

In some embodiments, an anti-HSV antibody as described herein is administered to a subject in need thereof in the incubation period (an early/asymptomatic stage). Typically, the incubation period for HSV-1 and HSV-2 before symptoms occur is from 2 to 12 days. Therefore, in certain examples, an anti-HSV antibody as described herein is administered to a subject in need thereof within 10 days, such as 10 days, 9 days, 8 days, 7 days, 6 days, 5 days, 4 days, 3 days, 2 days or 1 day post infection.

In some embodiments, an anti-HSV antibody as described herein is administered to a subject in need thereof after symptoms occur. In certain examples, an anti-HSV antibody as described herein is administered to a subject in need thereof after symptoms occur during the primary or initial outbreak. In certain examples, an anti-HSV antibody as described herein is administered to a subject in need thereof after symptoms occur during the recurrent period. In certain examples, an anti-HSV antibody as described herein is administered to a subject in need thereof after 10 days or later, such as 12 days, 15 days, 20 days, 25 days, 30 days, or more post infection.

In some embodiments, an anti-HSV antibody as described herein is administered to a subject in need thereof in a low frequency. In some embodiments, an anti-HSV antibody as described herein is administered every week or less frequently, for example, every 2 weeks, every 3 weeks, every 4 weeks, every 5 weeks, every 6 weeks, every 7 weeks, every 8 weeks or less frequently. In some embodiments, an anti-HSV antibody as described herein is administered once during a period of 1 to 3 months, for example, once per month, once every two months or once every three months.

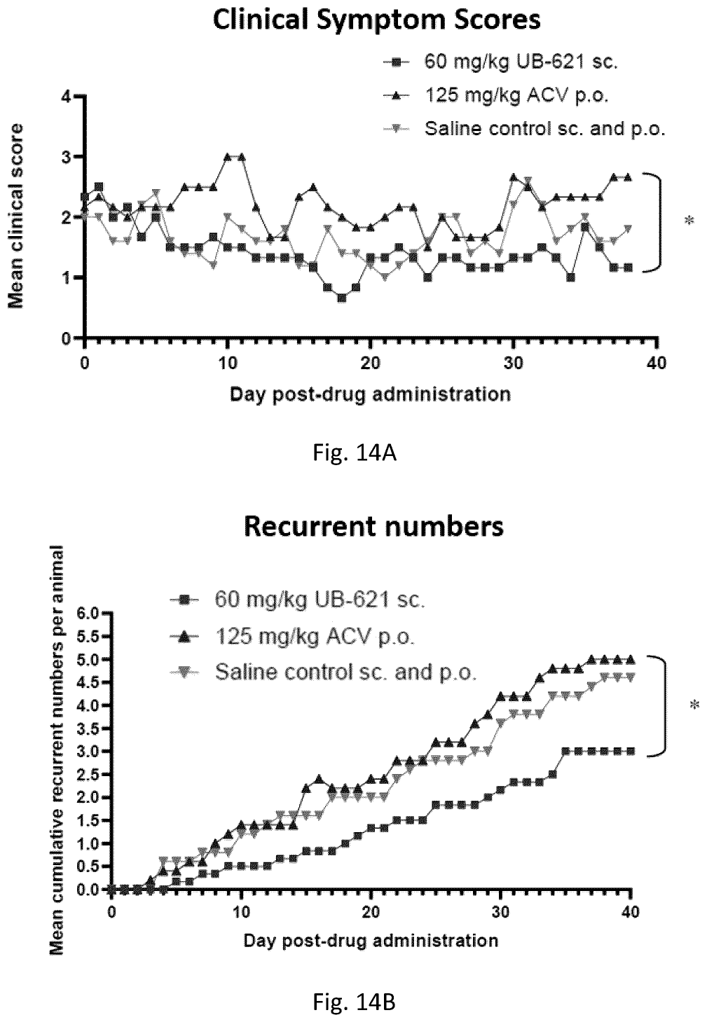

In some embodiments, the administration of an anti-HSV antibody as described herein is effective in decreasing the number of recurrence or frequency. In some examples, the recurrent numbers are decreased to 3 or below after 30 days post administration by a single dose of an anti-HSV antibody during the recurrent period compared to the number of recurrence over 3 (e.g. 4 or 5) by administration of multiple doses of a small molecular antiviral. (see FIG. 14B).

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

### Example 1: Determination of Binding Affinity of UB-621 to Recombinant gD by Surface Plasmon Resonance (SPR) Analysis

Kinetic assay of UB-621 (anti-HSV-gD mAb) binding to the recombinant gD was performed by Surface Plasmon Resonance (SPR) technology using Biacore X100 instrument. Anti-human IgG Fc fragment antibodies were first immobilized by amine coupling to the surface of the CMS gold sensor chip. UB-621 was diluted to 5 mg/mL in running buffer (two-fold serial dilutions, 6.25 to 100 nM), and was captured by an anti-FC antibody on a CMS chip. The gD samples diluted in HBS-EP buffer were injected, including the reference cell. The dissociation rate constant (kd), the association rate constant (ka), and the dissociation equilibrium constant (KD) were automatically calculated using a 1:1 model and steady-state affinity using Biacore Evaluation software.

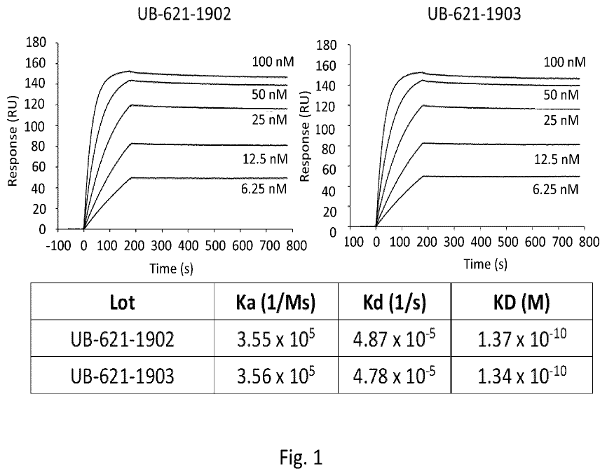

The mean KD of UB-621 is estimated to be 1.35×10⁻¹⁰ M (FIG. 1).

### Example 2: Neutralization of UB-621 Against HSV-1 and HSV-2 Laboratory Strain Infection

The potency of UB-621 against HSV infection was first studied in Vero cells by cell viability assay and expressed in IC₅₀ and IC₉₀. Four laboratory HSV strains tested were HSV-1 KOS, HSV-1 RE, HSV-2 333, and HSV-2 G. The viral strains were mixed at room temperature with UB-621 at serial concentrations for one hour. Then, the mixtures were aliquoted into a 96-well plate seeded with Vero cells. After incubation for 48 hours at 37° C., the plate was washed with PBS once, and the viable cells were incubated with 1% Alamar blue for 1 hour. Measure the fluorescence at 530 nm and 590 nm to calculate the cell death percentage.

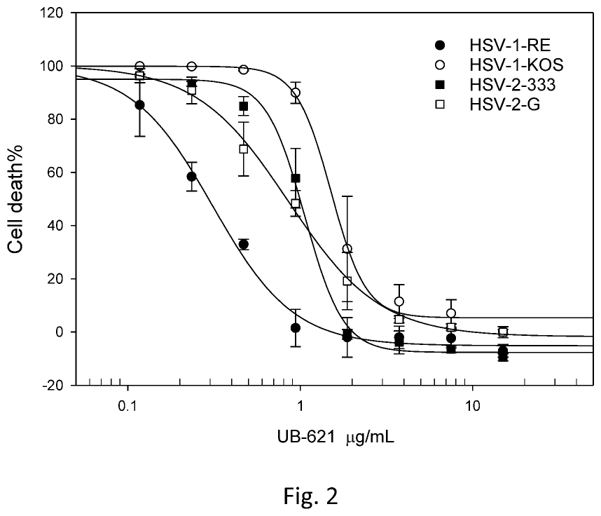

UB-621 completely neutralized all laboratory HSV-1 and HSV-2 strains (FIG. 2) with high potency, as shown in low levels of IC₅₀ and IC₉₀ (Table 2).

TABLE 2
UB-621 inhibits infection of laboratory HSV-1 and HSV-2
strains to Vero cells with low IC₅₀ and IC₉₀ values

| | HSV-1 RE | HSV-1 KOS | HSV-2 333 | HSV-2 G |
|---|---|---|---|---|
| IC₅₀ | | | | |
| μg/mL | 0.31 | 1.51 | 1.06 | 0.85 |
| nM | 2.07 | 10.07 | 7.07 | 5.67 |
| IC50 | | | | |
| μg/mL | 0.31 | 1.51 | 1.06 | 0.85 |
| nM | 2.07 | 10.07 | 7.07 | 5.67 |

### Example 3: The Therapeutic Effects of UB-621 on Genital HSV-2 Infection

3.1 Post-Infection Treatment Prior to the Appearance of Infection Symptoms

Female BALB/c mice of 6-8 weeks old were used for the HSV-2 intravaginal infection model (Marshak J O, et al., 2014). Mice were randomly assigned to hIgG control group and UB-621 treated group (n=6). On days 4 and 1 prior to HSV-2 intravaginal infection, the mice were subcutaneously injected with 2.5 mg of progesterone in the upper back. The mice were then inoculated intravaginally with 10 μL of virus suspension (HSV-2 strain 333, 1×10⁵ PFU). On day 1 post-infection, 300 μg of UB-621 (UB-621 treated group) or an equal amount of human IgG (hIgG control group) were administered to each mouse by subcutaneous injection. Clinical signs of genital infection were scored on days 1, 2, 3, 4, 5, 6, 7, 8, 10, and 12 post-infection, according to a composite 0-to-5 scale (Gill N, et al., 2005): 0, no sign of infection; 1, slight redness of external genitalia; 2, swelling and redness of external genitalia, and/or pus/mucus; 3, severe swelling of external genitalia with pus/mucus and some alopecia in the surrounding area; 4, ulceration of genital tissue, redness, and swelling; 5, increased ulceration, redness, swelling, hind limb paralysis; 6, death.

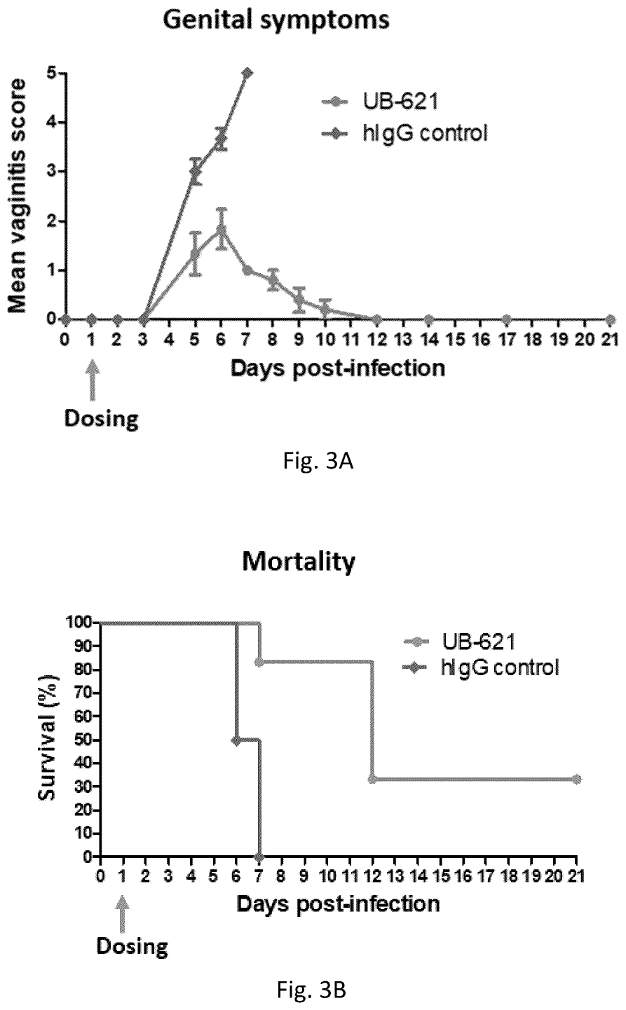

A single 300 μg dose of UB-621 controlled the genital symptoms and signs within scores 2, compared to score 5 in hIgG treated mice on day 7 post-HSV-2 infection (FIG. 3A). UB-621 treated mice prolonged survival time of 5.5 days in comparison to hIgG treated mice (12 days vs. 6.5 days). (FIG. 3B).

3.2 Post-Infection Treatment after the Appearance of Infection Symptoms

Mice were randomly assigned to the hIgG control group and UB-621 treated group (n=3). On days 4 and 1 prior to HSV-2 intravaginal infection, the mice were subcutaneously injected with 2.5 mg of progesterone in the upper back. The mice were then inoculated intravaginally with 10 μL of virus suspension (HSV-2 strain 333, 1×10⁵ PFU). On day 4 post-infection, 300 lig of UB-621 (UB-621 treated group) or an equal amount of human IgG (hIgG control group) were administered to each mouse by subcutaneous injection.

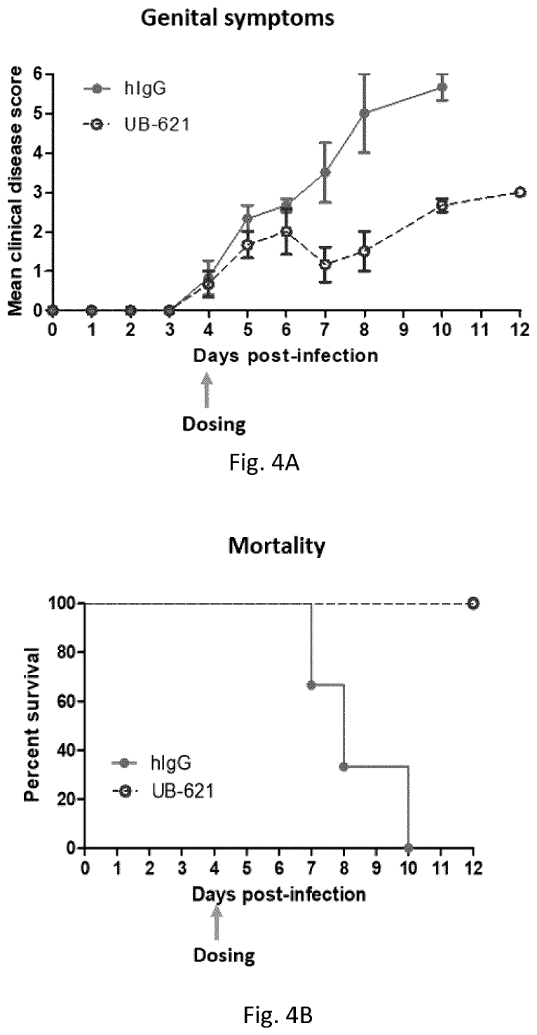

A single 300 μg dose of UB-621 controlled the genital symptoms and signs in score 3 compared to score 5 to 6 in hIgG treated mice on day 12 post-HSV-2 infection (FIG. 4A). UB-621 treated mice were 100% survived in comparison to hIgG treated mice with a median survival time of 8 days (FIG. 4B).

### Example 4: Neutralization of UB-621 Against HSV-1 RE TKnull and HSV-2 333 TKnull Mutant Strains in Vero Cells

A comparative UB-621-vs.-acyclovir (ACV) study was conducted using Vero cells infected with two HSV laboratory mutant strains, and the inhibition activity was analyzed by plaque assays. The wild-type HSV-1 RE and HSV-2 333 strains were mutated by inserting an enhanced green fluorescence protein (EGFP) in the TK gene so that the resultant HSV-1 RE TKnull and HSV-2 333 TKnull mutants are resistant to ACV. The Vero cells seeded in a 12-well plate were infected with the HSV-1 RE mutant or HSV-2 mutant (MOI=0.01) for 75 minutes at 37° C. The infected cells were then cultured for 24 hours in the medium with or without UB-621 or ACV at serial concentrations. The viral plaques were counted, and the half-maximum inhibitory activity expressed as IC₅₀ was calculated.

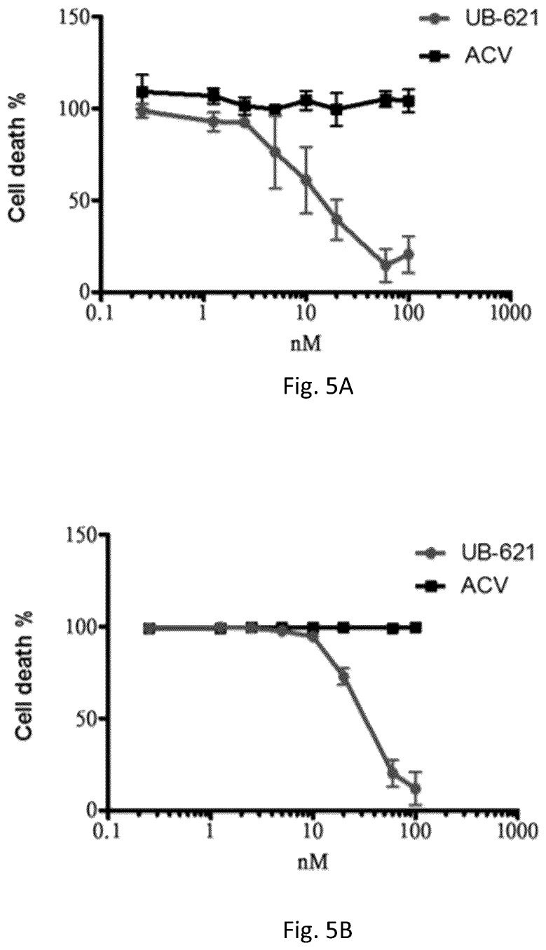

UB-621 neutralized both viral strains at a much higher potency over ACV (FIGS. 5A and 5B). UB-621 inhibited the infection with a low level of IC₅₀, at 11.3 nM for HSV-1 RE mutant and 28.1 nM for HSV-2 333 mutant (Table 3). The IC₅₀ values of UB-621 may represent a potency 1000-fold higher than by ACV.

TABLE 3
UB-621 inhibits both HSV-1 RE TKnull and HSV-2 333 TKnull
strains with IC₅₀ values much lower than by ACV (acyclovir)

| HSV (IC₅₀) | UB-621 | ACV | Relative potency |
|---|---|---|---|
| HSV-1 RE TKnull | 11.3 nM | >100 nM | NA |
| HSV-2 333 TKnull | 28.1 nM | >100 nM | NA |

### Example 5: Neutralization of UB-621 Against Clinically Derived HSV-1 and HSV-2 Isolates

5.1 American and Asian Origins and ACV Resistance.

The potency of UB-621 and ACV in inhibition of clinically derived HSV-1 and HSV-2 isolates was compared against 11 isolates of American, Asian origins, and acyclovir resistance. Four American isolates (HSV-1 Bethesda and HSV-1 RH; HSV-2 Bethesda and HSV-2 RH), 3 Asian isolates (HSV-2 JA-1, HSV-2 JA-2, and HSV-2 JA-3), and 4 isolates with ACV resistance (poly-mut, C7, C8, and IC) were investigated via a cell viability assay. The viral strains were mixed with UB-621 or ACV at serial concentrations at room temperature for one hour. Then, the mixtures were aliquoted into a 96-well plate seeded with Vero cells. After incubation for 48 hours at 37° C., the plate was washed with PBS once, and the viable cells were incubated with 1% Alamar blue for 1 hour. Measure the fluorescence at 530 nm and 590 nm to calculate the cell death percentage.

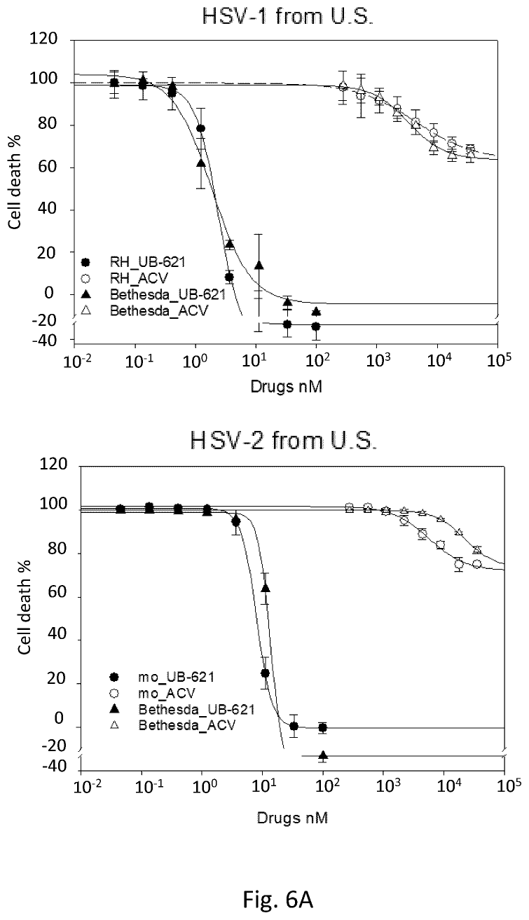

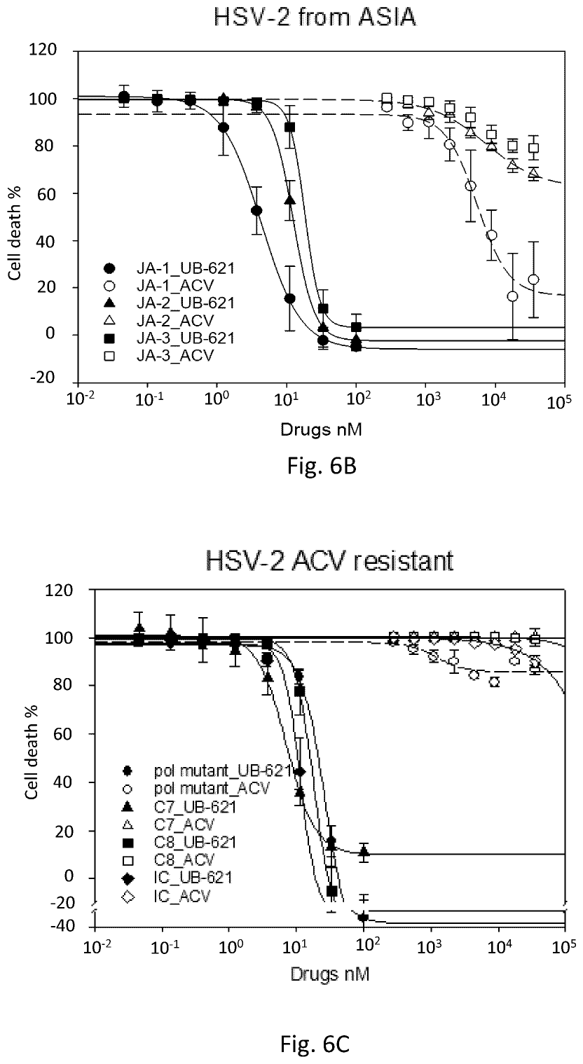

UB-621 neutralized all clinical HSV-1 and HSV-2 strains (FIGS. 6A, 6B and 6C) at a high potency (low IC₅₀ and IC₉₀) that were at least 500-fold greater than ACV (Table 4).

TABLE 4
UB-621 inhibits clinically derived HSV isolates of American
and Asian origins, and ACV-resistance, with IC50 and
IC90 values much lower than by ACV (acyclovir)

| | Clinical HSV isolates | | UB-621 | ACV | UB-621 | ACV |
|---|---|---|---|---|---|---|
| Types | Labeling | Other information | IC₅₀ (nM) | | IC₉₀ (nM) | |
| HSV-1 | RH | US origin | 2.38 | 4,730 | 6.14 | >35,500 |
| | Bethesda | | 1.87 | 3,620 | 9.52 | >35,500 |
| HSV-2 | MO | | 8.05 | 6,110 | 15.25 | >35,500 |
| | Bethesda | | 13.6 | >35,500 | 22.4 | >35,500 |
| | JA-1 | Asian origin | 4.31 | 5,530 | 18.2 | >35,500 |
| | JA-2 | | 12.7 | 7,970 | 26.67 | >35,500 |
| | JA-3 | | 18.3 | 6,310 | 31.6 | >35,500 |
| | poly-mut | polymerase mutant | 27.1 | >35,500 | 69.79 | >35,500 |
| | C7 | TK mutant | 7.14 | >35,500 | 20.85 | >35,500 |
| | C8 | | 17.9 | >35,500 | 36.2 | >35,500 |
| | IC | ACV-resistant | 11.4 | >35,500 | 20.85 | >35,500 |

5.2 German Origin and Other Drug Resistance.

UB-621 at serially-diluted concentrations from 250 to 0 nM were incubated in cell culture medium for 1 hour at 37° C. with drug-resistant HSV-1 or HSV-2 isolates at a viral load of 100 TCID₅₀. The antibody virus inoculum was then applied to Vero cell monolayers grown in 96-well plates. The cytopathic effect (CPE) was scored after incubation for 48 hours by light microscopy. The antibody concentration required for complete neutralization of the virus was defined as the neutralizing titer.

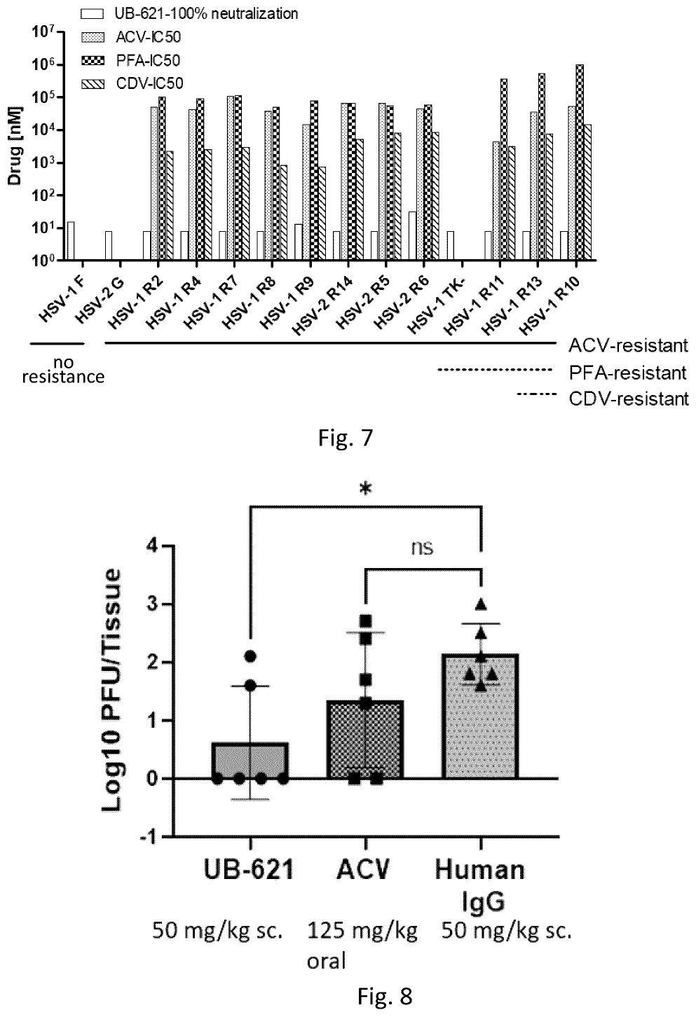

UB-621 could completely neutralize all tested drug-sensitive or single- or multi-drug resistant clinical HSV isolates (HSV-1, including R2, R4, R7, R8, R9, R10, R11, and R13) and HSV-2 isolates (R5, R6, and R14) at a remarkably low concentration ranging from 7.8 to 31.3 nM, in comparison to those by the small molecular antivirals that inhibit HSV at IC₅₀ levels of low-to-high μM (FIG. 7). The neutralization efficacy of UB-621 was independent of viral strain resources and resistance statuses.

### Example 6: The Efficacy Study of UB-621 Against the Infection of the ACV-Resistant HSV-1 Clinical Isolate in Mice

BALB/c mice were divided into 3 groups with 6 mice per group. After anesthetizing each mouse, the cornea of the right eye in each mouse was scarified with a needle, and 5 μL of virus inoculum containing 5×10⁶ pfu of ACV-resistant HSV-1 clinical isolate was applied. One dose of UB-621 (50 mg/kg) or human IgG (50 mg/kg) was administered to each designated mouse by subcutaneous injection on day 1 post-infection. Acyclovir (ACV) at 125 mg/kg was administered to each designated mouse by oral gavage twice daily on days 1 and 2 post-infection. All mice were sacrificed on day 3 post-infection, and the mouse right eyes, trigeminal ganglia, and brains were harvested to determine the viral loads in tissues by plaque assays.

In comparison with the human IgG (control) treatment, UB-621 at 50 mg/kg significantly reduced the virus replication in the eyes (the virus inoculation site) (FIG. 8). There was no significant difference between the ACV-treated group and the control group. However, this HSV clinical isolate strain was difficult to transmit and replicate in all mice's neural tissues (trigeminal ganglia and brains), so the differences in drug efficacy in these tissues were not assessed.

### Example 7: The Efficacy Studies of UB-621 Against the Infection of ACV-Resistant HSV-2 Clinical Isolates in Mice

7.1 HSV-2 Clinical Isolate IC Strain

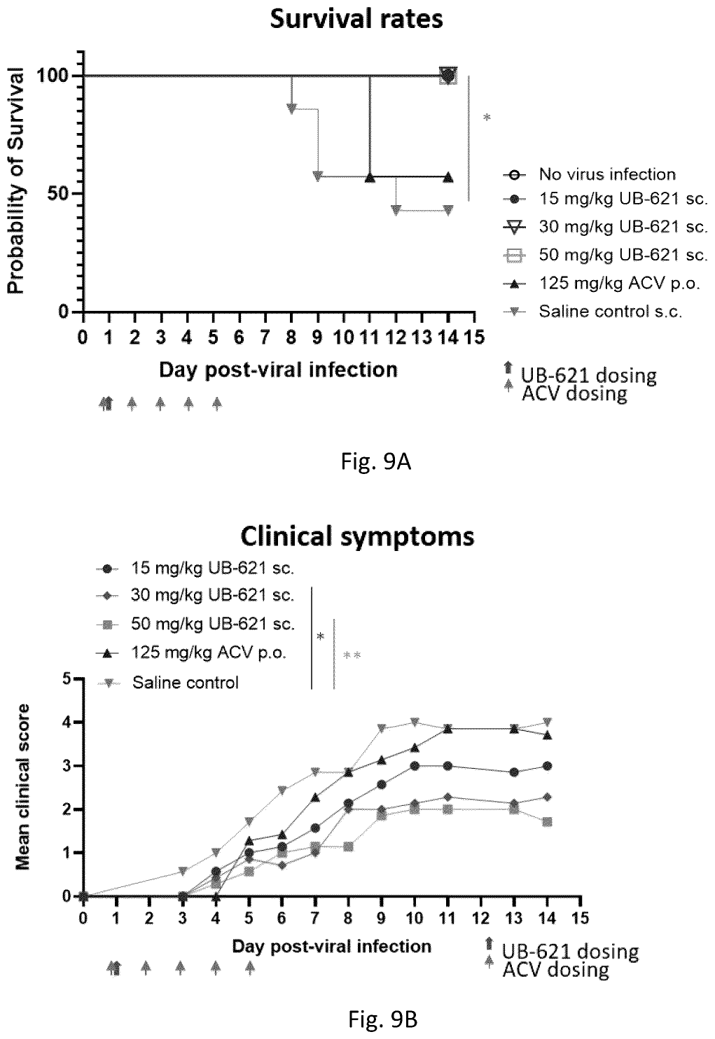

Female BALB/c mice of 6-8 weeks old were given 0.1 ml of a suspension containing 2.5 mg progesterone by subcutaneous injection on days 4 and 1 before the challenge to increase susceptibility to vaginal HSV infection as previously reported. Animals were then intra-vaginally inoculated with 10 μL of a suspension containing 1×10⁶ pfu of the HSV-2 clinical isolate IC strain (FIG. 9). Animals were administered subcutaneously with a single dose of UB-621 on day 1 post-infection, or orally with multiple doses of ACV on days 1-5. Animals were then followed daily for at least 15 days. The severity of vaginitis was scored by the composite scale from 0 to 6.

UB-621 on the dose levels from 15 mg/kg to 50 mg/kg fully protected mice from death caused by virus infection (FIG. 9A). A single dose of UB-621 significantly controlled the genital symptoms in dose-dependency compared to saline control mice and ACV-treated mice (FIG. 9B).

7.2 HSV-2 Clinical Isolate Pol-Mut Strain

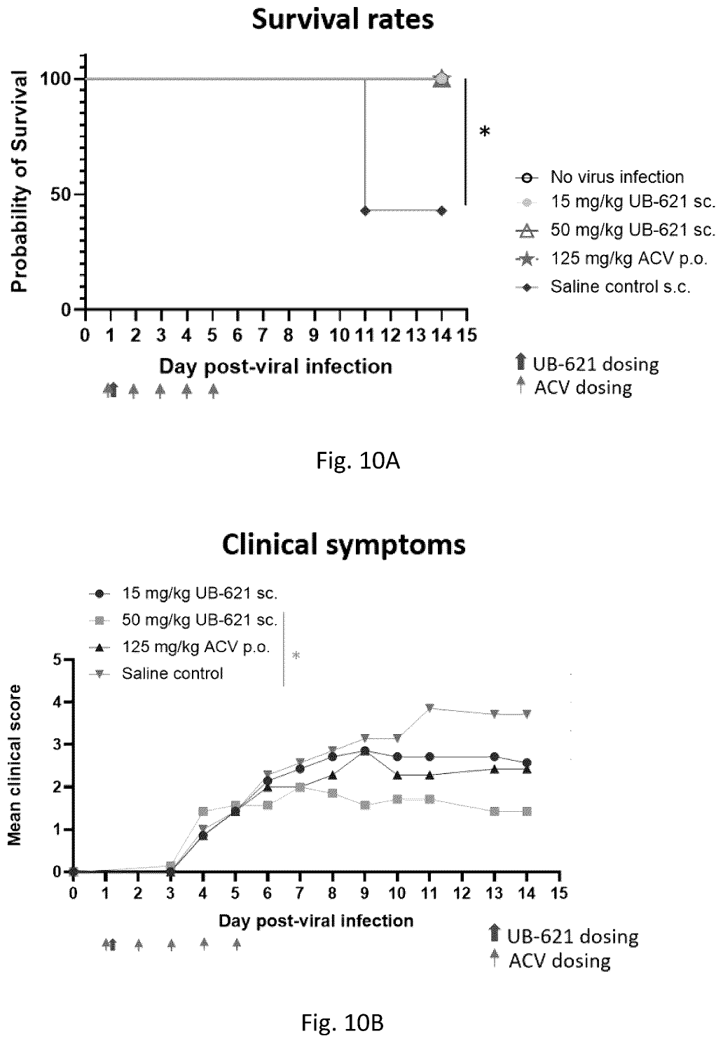

A separate study was performed using the other clinical isolate pol-mut strain. In brief, animals were intra-vaginally inoculated with 10 μL of a suspension containing 1×10⁶ pfu of the HSV-2 clinical isolate pol-mut strain (FIG. 10). Animals were administered subcutaneously with a single dose of UB-621 on day 1 post-infection, or orally with multiple doses of ACV on days 1-5. Animals were then followed daily for at least 15 days.

Similar results were observed in the study of UB-621 efficacy evaluation on the clinical isolate pol-mut strain. UB-621 on the dose levels from 15 mg/kg to 50 mg/kg fully protected mice from death (FIG. 10A). A single dose of UB-621 significantly controlled the genital symptoms in dose-dependency compared to saline control mice (FIG. 10B).

### Example 8: The Inhibition of HSV-1 and HSV-2 Cell-to-Cell Spread by UB-621

The inhibition of cell-to-cell spread by UB-621 was investigated as previously described (Krawczyk A, et al., 2013) with modifications. Briefly, Vero cells were seeded on 24-well plates at 1×10⁵ cells/well. The confluent cell cultures were infected with 200 TCID₅₀ HSV-1- or HSV-2-AgE-GFP reporter virus/well. After 2 hours of incubation, the inoculation medium was removed, and the cell cultures were incubated with serial dilutions of UB-621 (0-1000 nM). After 2 days of incubation, the plaque formation was examined by fluorescence microscopy. The 2% DMEM only was used as a negative control, and the anti-gB mAb H1817 (Pereira L, et al., 1989) was used as a positive control. Plaque formation was assessed by fluorescence microscopy.

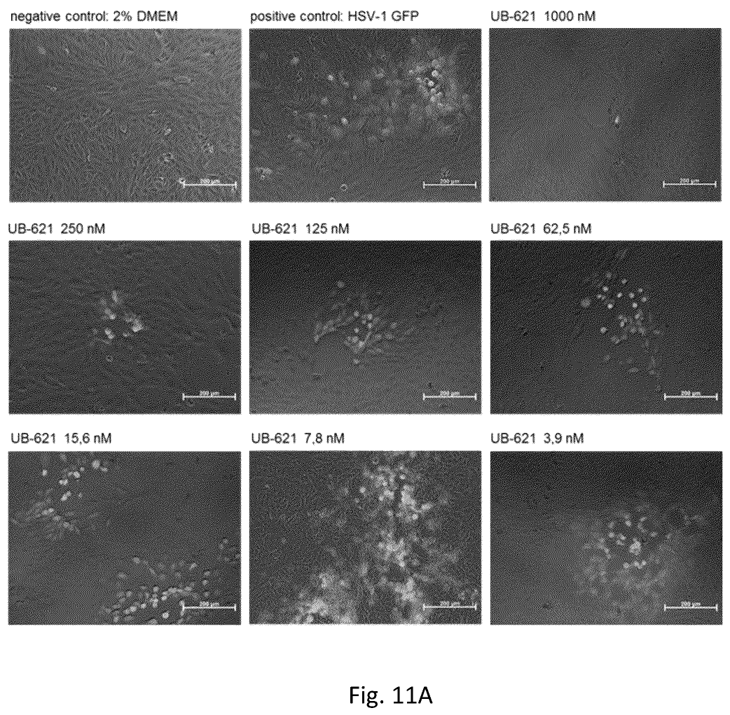

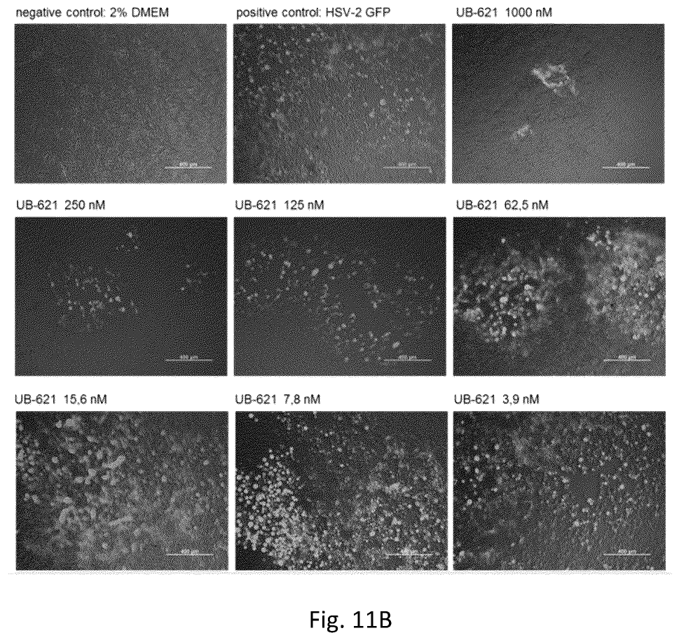

Plaque formation by HSV-1 and HSV-2 decreased with increasing UB-621 concentrations. Complete neutralization of HSV-1 was observed at a UB-621 concentration of 1000 nM (150 μg/mL) (FIG. 11A). UB-621 clearly shows plaque-reducing effects against HSV-2, which is evidence for cell-to-cell spread inhibiting activity (FIG. 11B).

### Example 9: Inhibition of HSV-1 Anterograde Neuron-to-Neuron Transmission in BALB/c Mice by UB-621

Female BALB/c mice were infected in the right eye via corneal inoculation with HSV-1 RE strain at 1×10⁶ pfu in 5 μL per mouse. The animals received a single intraperitoneal dose of UB-621 at 15 mg/kg on day 1 after infection. The animals, 5 per group (n=5), were sacrificed on day 5 post-infection for analysis of viral load in the right eye, right trigeminal ganglion, and the whole brain.

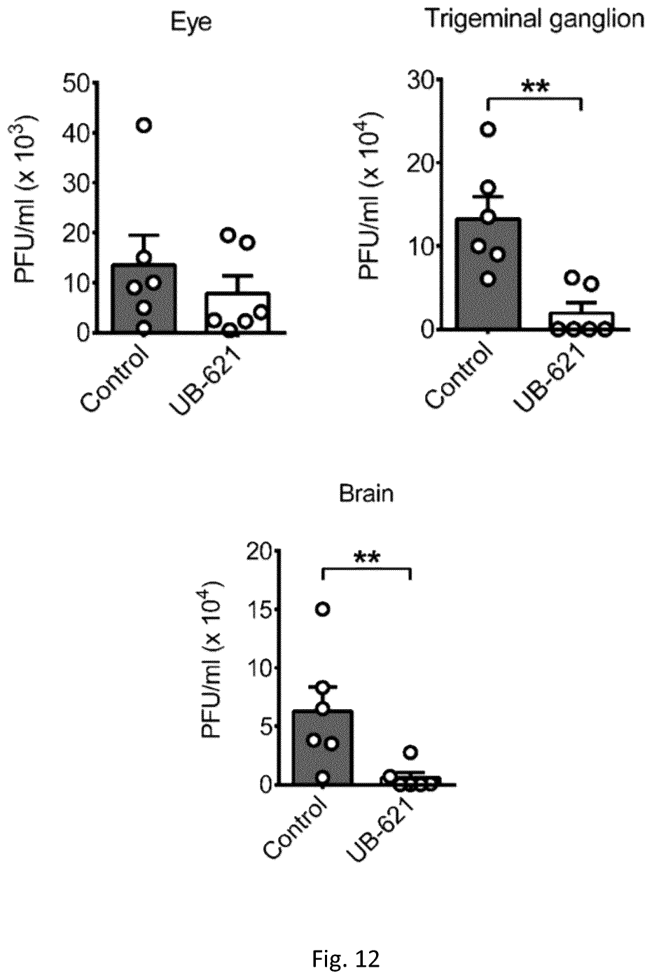

The results indicated that the presence of UB-621 can significantly suppress neuron-to-neuron transmission from the eye to the trigeminal ganglion and brain (FIG. 12).

### Example 10: The Efficacy Study of UB-621 on Recurrent HSV-2 Infection in Guinea Pigs

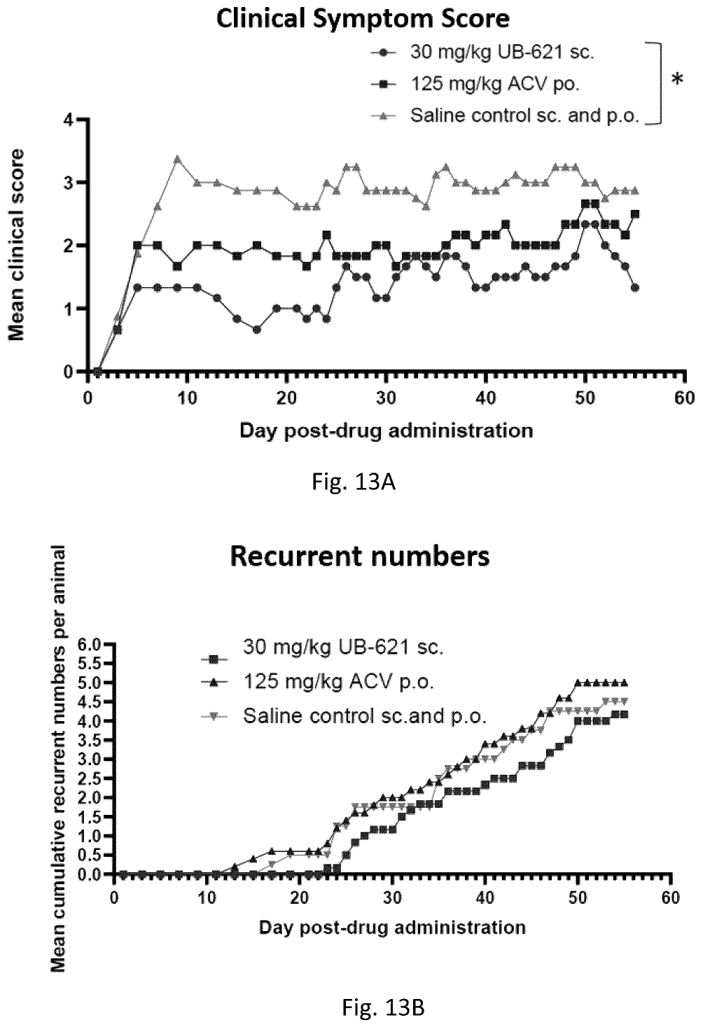

The guinea pig genital model, unlike mouse models, manifests both acute disease and spontaneous recurrences. Female Hartley strain guinea pigs (200-300 g) were infected with 10⁶ PFU of HSV-2 strain 333 in the genital tract on day 0. To study the UB-621 efficacy and the effects on recurrence (FIGS. 13A and 13B, and FIGS. 14A and 14B) when dosed in HSV primary and recurrent infection, a single dose of UB-621 (30 or 60 mg/kg) was administered subcutaneously on day 1 or day 20 post-infection. Multiple doses of Acyclovir (125 mg/kg, twice daily) were administered orally on days 1-7 or days 20 to 26 post-infection. Control animals were dosed with normal saline by both subcutaneous and oral routes. The severity of post-infection symptoms was determined by direct examination of external genital skin and animal activities. Each animal was observed daily for 55 days (dosing in the acute infection period; FIG. 13A) or for 40 days after treatment (dosing in the recurrent infection period; FIG. 14A) by use of a clinical scale of 0 for no lesions, 1 for erythema only, 2 for single or few vesicles, 3 for ulcerated lesions, and 4 for abnormal movement in addition to lesions at score 3. Animals were humanitarian sacrificed when symptoms at score 4. Recurrence was identified when the clinical symptom was more severe than the previous day.

The results demonstrated that a single dose of UB-621 relieved vaginitis clinical symptoms effectively whether dosing the infected animals in the primary infection (FIG. 13A) or after the first HSV recurrence (FIG. 14A). UB-621 treatment not only delayed incidences of recurrence but decreased the cumulative numbers of recurrence in comparison to treatment with saline or ACV (FIGS. 13B and 14B).

### 3. Conclusion

UB-621 is potent to neutralize wild-type and drug-resistant HSV-1 and HSV-2 (Examples 2, 4, and 5) as well as reducing the disease symptoms and increasing the survival rates in HSV-infected mice and guinea pigs by a single dose of UB-621 (Examples 3, 6, 7, 9 and 10).

UB-621 inhibits HSV in vitro replication effectively in all 23 drug-resistant HSV clinical isolates, where the isolates are resistant to several small molecule drugs (e.g., ACV, PFA) (Example 5).

Many patients suffer from drug-resistant HSV infection and need to use higher doses of small molecule drugs to treat the disease. While a single dose of UB-621 can reduce the disease symptoms and increase the survival rates in primary HSV infection with drug-resistant strains (Examples 6 and 7).

Some patients may develop recurrent symptoms of HSV when patients' immune systems are weakened, or small molecule drug therapy is interrupted. A single dose of UB-621 at the primary HSV infection phase shows strong therapeutic effects in the HSV recurrent guinea pig model (Example 10).

The significantly therapeutic effects of UB-621 in animals can be related to the neutralization of HSV gD and the inhibition of HSV cell-to-cell transmission (Examples 8 and 9).

There are no approved mAb drugs for HSV treatment so far, and there are many limitations and side effects of small molecule drugs. UB-621 shows potent therapeutic efficacy in animal models and can benefit patients infected with drug-resistant HSV strains and poor symptom control with small molecule drugs.

### REFERENCES

Boado R J, Zhou Q-H, Lu J Z, et al. Pharmacokinetics and brain uptake of a genetically engineered bifunctional fusion antibody targeting the mouse transferrin receptor. Mol Pharmacol 7:237-244 (2010).
Eddie C hang, Laurence Galle, David Maggs, D. Mark Estes, and William J. Mitchell. Pathogenesis of Herpes Simplex Virus Type 1 Induced Corneal Inflammation in Perforin Deficient Mice. (2000) Journal of Virology. 74: 11832 11840.
Gill N, Rosenthal K L, Ashkar A A. NK and NKT cell-independent contribution of interleukin-15 to innate protection against mucosal viral infection. J. Virol. 79:4470-4478 (2005).
Krawczyk A, Arndt M A, Grosse-Hovest L, Weichert W, Giebel B, Dittmer U, Hengel H, Jager D, Schneweis K E, Eis-Hubinger A M, Roggendorf M, Krauss J. Overcoming drug-resistant herpes simplex virus (HSV) infection by a humanized antibody. PNAS, USA 110:6760-6765 (2013).
Marshak J O, Dong L, Koelle D M, et al. The Murine Intravaginal HSV-2 Challenge Model for Investigation. Methods Mol Biol. 1144:305-327 (2014).
Pereira L, Ali M, Kousoulas K, Huo B, Banks T. Domain structure of herpes simplex virus 1 glycoprotein B: neutralizing epitopes map in regions of continuous and discontinuous residues. Virology 172:11-24 (1989).
Pepinsky R B, Shao Z, Ji B, et al. Wang, Q., Meng, G., Walus, L., Lee, X., Hu, Y., Graff, C., Garber, E., Meier, W., and Mi, S. Exposure levels of anti-LINGO-1 Li81 antibody in the central nervous system and dose-efficacy relationships in rat spinal cord remyelination models after systemic administration. J. Pharmacol. Exp. Ther. 339:519-529 (2011).

1. A method for treating drug-resistant and/or recurrent herpes simplex virus (HSV) infection comprising administering to a subject in need thereof an anti-HSV antibody, wherein the anti-HSV antibody specifically binds to the glycoprotein D (gD) of herpes simplex virus-1 (HSV-1) and herpes simplex virus-2 (HSV-2).

2. The method of claim 1, wherein the anti-HSV antibody is effective in neutralizing drug-resistant HSV strains and/or inhibiting viral transmission.

3. The method of claim 1, wherein the anti-HSV antibody comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 4, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 9, a light chain complementary determining region (LC CDR2) comprising the amino acid sequence of SEQ ID NO: 11, and a light chain complementary determining region 3 (LC CDR3) comprising the amino acid sequence of SEQ ID NO: 13.

4. The method of claim 3, wherein
the VH comprises the amino acid sequence of SEQ ID NO: 15; and/or
the VL comprises the amino acid sequence of SEQ ID NO: 16.

5. The method of claim 1, wherein the anti-HSV antibody comprises
(a) a heavy chain variable region (VH) which comprises a heavy chain complementary determining region 1 (HC CDR1) comprising the amino acid sequence of SEQ ID NO: 18, a heavy chain complementary determining region 2 (HC CDR2) comprising the amino acid sequence of SEQ ID NO: 20, and a heavy chain complementary determining region 3 (HC CDR3) comprising the amino acid sequence of SEQ ID NO: 22; and
(b) a light chain variable region (VL) which comprises a light chain complementary determining region 1 (LC CDR1) comprising the amino acid sequence of SEQ ID NO: 24, a light chain complementary determining region (LC CDR2) comprising the amino acid sequence of SEQ ID NO: 26, and a light chain complementary determining region 3 (LC CDR3) comprising the amino acid sequence of SEQ ID NO: 28.

6. The method of claim 5, wherein
the VH comprises the amino acid sequence of SEQ ID NO: 30; and/or
the VL comprises the amino acid sequence of SEQ ID NO: 31.

7. The method of claim 1, wherein the anti-HSV antibody comprises
(a) a heavy chain variable region (VH) which comprises a heavy chain complementary determining region 1 (HC CDR1) comprising the amino acid sequence of SEQ ID NO: 2, a heavy chain complementary determining region 2 (HC CDR2) comprising the amino acid sequence of SEQ ID NO: 33, and a heavy chain complementary determining region 3 (HC CDR3) comprising the amino acid sequence of SEQ ID NO: 6; and
(b) a light chain variable region (VL) which comprises a light chain complementary determining region 1 (LC CDR1) comprising the amino acid sequence of SEQ ID NO: 35, a light chain complementary determining region (LC CDR2) comprising the amino acid sequence of SEQ ID NO: 11, and a light chain complementary determining region 3 (LC CDR3) comprising the amino acid sequence of SEQ ID NO: 38.

8. The method of claim 7, wherein
the VH comprises the amino acid sequence of SEQ ID NO: 40; and/or
the VL comprises the amino acid sequence of SEQ ID NO: 41.

9. The method of claim 1, wherein the antibody is an antigen-binding fragment thereof.

10. The method of claim 1, wherein the antibody is humanized.

11. The method of claim 1, wherein the subject is susceptible to or infected with a drug-resistant HSV strain.

12. The method of claim 1, wherein the subject is a patient having a weakened immune system.

13. The method of claim 1, wherein the anti-HSV antibody is administered to the subject in a single dose.

14. The method of claim 1, wherein the anti-HSV antibody is administered to the subject at an early stage of the infection.

15. The method of claim 1, wherein the antibody is administered in an amount effective in reducing symptoms caused by the HSV infection.

16. The method of claim 1, wherein the antibody is administered in an amount effective in delaying recurrent incidences and/or reducing recurrent frequency.

17. The method of claim 1, wherein the anti-HSV antibody is administered to the subject after symptoms occur.

18. An anti-HSV antibody as defined in claim 1 or a composition thereof for use in treating drug-resistant and/or recurrent HSV infection in a subject in need thereof.

19. (canceled)

20. An anti-HSV antibody or a composition thereof for use of claim 18, wherein
the subject is susceptible to or infected with a drug-resistant HSV strain;
the subject is a patient having a weakened immune system;
the anti-HSV antibody is administered to the subject in a single dose;
the anti-HSV antibody is administered to the subject at an early stage of the infection;
wherein the antibody is administered in an amount effective in reducing symptoms caused by the HSV infection;
wherein the antibody is administered in an amount effective in delaying recurrent incidences and/or reducing recurrent frequency; and/or
wherein the anti-HSV antibody is administered to the subject after symptoms occur.
